(19) United States
(12) Patent Application Publication
(10) Pub. No.: US 2004/0198829 A1
(43) Pub. Date: Oct. 7, 2004

(54) Prostanoids augment ocular drug penetration

(76) Inventors: William Eric Sponsel, San Antonio, TX (US); Randolph D. Glickman, San Antonio, CA (US); Gianmarco Paris, San Antonio, TX (US); Vanessa Bernal, San Antonio, TX (US); John Graybill, San Antonio, TX (US)

Correspondence Address: Shelly P M Fussey Williams, Morgan & Amerson, Suite 1100, 10333 Richmond, Houston, TX, 77042, US

(21) Appl. No.: 10/476,090

(22) PCT Filed: Apr. 23, 2002
(86) PCT No.: PCT/US02/13057

Related U.S. Application Data
(60) Provisional application No. 60/285,856, filed on Apr. 23, 2001.

(51) Int. Cl. A61K 31/557

(52) U.S. Cl. 514/573

(57) ABSTRACT

Disclosed is the surprising discovery that prostanoids function to effectively increase the transport of therapeutic agents into the eye. The invention thus provides new methods, combinations, formulations, compositions and kits for prophylactic and therapeutic intervention in various ocular diseases, disorders and infections and in combined use with surgical intervention.

The present application claims priority to U.S. provisional application Serial No. 60/285,856, filed Apr. 23, 2001, the entire text, figures and claims of which application are incorporated herein by reference without disclaimer.

### BACKGROUND OF THE INVENTION

1. Field of the Invention

The present invention relates generally to the field of ocular therapeutics. More particularly, it concerns the discovery that prostanoids are effective to increase the transport of therapeutic agents into the eye. The invention therefore provides new methods, combinations, formulations, compositions and kits for the prevention and/or treatment of various ocular diseases, disorders and infections and in combined use with surgical procedures.

2. Description of Related Art

There are a large number of diseases, disorders and infections that significantly impair vision in humans and animals. As this area has been the subject of intense biomedical and clinical research for some time, a number of effective biological agents are now available for therapeutic intervention in ocular diseases and infections and for use in combination with surgical procedures. Despite the progress made, many harmful conditions are still unfortunately widespread and continue to exert a significant toll in human suffering and economic terms.

Some of the more common ocular therapeutics currently approved for administration to patients include agents for treating glaucoma and various antibiotics, the latter of which are often used in conjunction with cataract and corrective surgical procedures. Unfortunately, the effectiveness of even the most potent drugs is often limited due to inadequate penetration. In fact, treatment with various drugs is frequently limited on such grounds, particularly where the chemistry of the therapeutic agents renders them less permeable by nature and/or when using drugs of relatively large molecular weight.

Particular examples of available agents that are nonetheless limited in their clinical application are antibiotics, including anti-fungal agents. Ocular preparations of such drugs have limited efficacy due to poor topical ocular permeability. Even when administered systemically, many of the available drug formulations are relatively ineffective due to limited permeability across the blood-retinal barrier. This is a significant drawback to the use of such agents before and after ocular surgery, and in attempts to treat resistant ocular bacterial and fungal infections.

Therefore, there remains in the art an evident need for new and more effective means for treating ocular diseases and infections. Within this general desire, the identification of components or methodology to improve the effectiveness of clinically approved agents would be an important development, thus avoiding the variability and expense associated with drug development per se. The identification of agents capable of improving the effectiveness of a broad spectrum of ocular therapeutics would be a particularly important advance in the field. Despite presumed attempts to make progress in such areas, new compositions and methods to address such drawbacks in the art are still urgently needed.

### SUMMARY OF THE INVENTION

The present invention addresses the foregoing and other long-felt needs in the art by providing new methods, combinations, formulations, compositions and kits for improving the transport of therapeutic agents, particularly transport into the eye. The invention is based, in large part, upon the surprising discovery that prostanoids effectively increase the transport and/or penetration of therapeutic agents into the eye. The new methods, combinations, formulations, compositions and kits of the invention are therefore useful in combination with therapeutic agents, particularly ocular therapeutics, for example, in the prevention and/or treatment of various ocular diseases, disorders and infections and in combined use before and after a range of surgical procedures.

The claimed invention was developed from the inventors' reasoning that prostanoids, such as prostaglandins, which are known to increase aqueous humor outflow and thereby decrease intraocular pressure, would potentiate the ocular penetration of drugs applied externally. Although now broadly applicable in light of the discoveries and teachings of this disclosure, the present invention was originally exemplified by the use of the topical prostaglandin analogue latanoprost (Xalatan™; Pharmacia & Upjohn) to increase the penetration and accumulation of antifungal agents, such as Voriconazole (Pfizer), in various ocular tissues.

The inventors' original idea that prostanoids and prostaglandins would potentiate the ocular penetration of therapeutic agents clearly runs contrary to the conventional wisdom in the art prior to the present invention. In addition to the new concept that prostanoids could transport, and/or increase the accumulation of agents in the eye, the practical demonstration of the successful use of the invention is important, given the complex phenomena operating in ocular transport and penetration. For example, aqueous humor egress itself is a complex phenomenon relying on many physiological and metabolic factors, and is not subject to osmotic or concentration gradients of solute between the intraocular and extraocular compartments. The present demonstration of significantly increased penetration in living animals is therefore important.

Although by no means limited by any theory of operation of the invention, the inventors contemplate that prostanoids could increase the transport and/or accumulation of agents in the eye by increasing the speed of uptake, amount and/or concentration of the substance coadministered with prostanoid or prostaglandin application to the eye. One potential mechanism for prostanoids increasing aqueous humor egress rates relies upon activation of a specific collagenase, which is believed to break down interstitial connective tissue between the fibers of the ciliary muscle, opening the so-called uveoscleral outflow pathway. This secondary aqueous outflow pathway typically accounts for less than 10% of outflow in human eyes. The present invention shows that prostaglandins can affect the changes with the ocular uveal system such that transport of drugs into the eye is facilitated.

The discoveries of the present invention are particularly important as they provide for a major new area of use for the already widely approved class of drugs: the prostanoids and prostaglandins. The invention has the further advantage in that the new methods, combinations, formulations, compositions and kits for preventing and/or treating various ocular diseases and infections permit the more effective use of biological agents that are already clinically approved for ocular administration. As such, new anti-microbial, anti-fungal and other therapeutic agents do not need to be developed, but rather the existing compounds can be used more widely and to better advantage.

The present invention thus provides new ophthalmically acceptable formulations, compositions, combinations and kits comprising one or more of a range of prostanoids or prostaglandins in combination with any agent intended for provision to the eye. The invention further provides a range of new prophylactic and therapeutic methods based upon the combined use of one or more prostanoids or prostaglandins with various ocular therapeutics in the prevention or treatment of essentially all ocular diseases, disorders, infections and in connection with all relevant surgical procedures.

In addition, due to the underlying transport phenomenon of the invention, as transport across the sclera of the eye has been demonstrated, the invention is also applicable to transport of therapeutic agents across other biological membranes and barriers, particularly across the blood brain barrier, e.g., to increase drug penetration and/or transport into the brain and cerebrospinal fluid. The invention thus provides a range of methods, combinations, formulations, compositions and kits for use in the transport of macromolecules, biological and therapeutic agents across biological membranes and barriers and into desired sites in the body.

In certain embodiments, the invention provides methods of transporting macromolecules, biological and therapeutic agents, drugs and such like through biological membranes and barriers, particularly “less permeable” or “transport-resistant” biological membranes and barriers, such as the sclera of the eye and blood brain barrier. Such methods generally comprise contacting the biological membrane, barrier, less permeable or transport-resistant biological membrane or barrier with at least a first macromolecule, biological or therapeutic agent or drug and an amount of at least a first prostanoid effective to transport the macromolecule, biological or therapeutic agent or drug across the biological membrane or barrier.

Methods of transporting macromolecules, biological or therapeutic agents or drugs into the eye are provided, which comprise contacting the eye with the macromolecule, biological or therapeutic agent or drug and the prostanoid in transport-effective amounts and under transport-effective conditions. Methods of transporting macromolecules, biological or therapeutic agents or drugs across the blood brain barrier are provided, which comprise contacting the blood brain barrier with the macromolecule, biological or therapeutic agent or drug and the prostanoid in transport-effective amounts and under transport-effective conditions.

The biological transport methods are conducted using “transport-effective amounts”, under “transport-effective conditions” and for “transport-effective times”, such that a detectable, and preferably, a biologically or therapeutically effective amount of the macromolecule, biological or therapeutic agent or drug is transported across the transport-resistant biological membrane or barrier, irrespective of the underlying mechanism. It is currently envisioned that the “transport-effective amounts” or “therapeutically effective doses” of prostanoids will be lower for ocular transport and that higher amounts or doses will be used in CNS transport, although this is by no means binding on the practice of the invention.

In methods of transporting macromolecules, agents or drugs into the eye, the macromolecule, agent or drug is therefore “transported” into the eye such that the macromolecule, agent or drug is present, and preferably present in a biologically or therapeutically effective amount, in at least a first intraocular compartment. In terms of transporting macromolecules, agents or drugs across the blood brain barrier, the macromolecule, agent or drug is “transported” across the blood brain barrier such that the macromolecule, agent or drug is present, and preferably present in a biologically or therapeutically effective amount, in at least a first compartment of the brain or central nervous system.

“Transport” across the transport-resistant membrane or barrier thus results in the presence of a detectable, and preferably, a biologically or therapeutically effective amount of the macromolecule, agent or drug in one or more biological tissues or biological spaces on the side of the membrane or barrier other than the side to which the macromolecule, agent or drug was applied. This is the meaning of “transported” in the context of the present invention, in that an increased amount, and preferably a biologically or therapeutically effective amount, of the macromolecule, agent or drug accumulates in the biological tissue or space on the side of a membrane or barrier other than the side to which the macromolecule, agent or drug is applied.

Thus, “transport” is used without necessarily meaning “active transport” in a strict biochemical context, but means “transport” simply in the sense that the macromolecule, agent or drug “penetrates” the transport-resistant biological membrane or barrier, such that it is “provided to the tissues or space on the other side”. Although the present studies in live animals are important to demonstrate the effectiveness of the invention, some aspects of the phenomena underlying the invention may be intrinsic to the tissues involved and not require intact circulation. Therefore, although the present application proposes certain mechanisms of action for the operation of the invention, those of ordinary skill in the art will understand that such mechanistic proposals are not limiting on the practice of the invention, which can be readily implemented without undue experimentation in light of the present disclosure (and without understanding any mechanism).

The invention thus provides methods of increasing the intraocular amount of a macromolecule, biological or therapeutic agent or drug, comprising providing to an eye of an animal or human at least a first macromolecule, agent or drug and an amount of at least a first prostanoid effective to increase the intraocular amount of the macromolecule, agent or drug. In ocular embodiments, increasing the “intraocular” amount includes increasing the amount in the periocular or intraocular space of the eye, in the aqueous and in the vitreous. Transport into the vitreous is particularly preferred and demonstrated herein.

Further provided are methods of increasing the intracerebral or cerebrospinal amount of a macromolecule, biological or therapeutic agent or drug, comprising providing to the blood brain barrier of an animal or human at least a first macromolecule, agent or drug and an amount of at least a first prostanoid effective to increase the intracerebral or cerebrospinal amount of the macromolecule, agent or drug.

Treatment methods of the invention include administering a transport-competent amount of at least a first prostanoid to a selected interior tissue an animal or human; and subsequently directly or indirectly providing to the selected interior tissue a therapeutically effective amount of at least a first therapeutic agent; wherein the prostanoid augments the accumulation of the therapeutic agent in the selected interior tissue, thereby providing treatment.

Ocular treatment methods include administering a transport-competent amount of at least a first prostanoid to an eye of an animal or human; and subsequently directly or indirectly providing to the eye a therapeutically effective amount of at least a first ocular therapeutic agent; wherein the prostanoid augments the accumulation of the ocular therapeutic agent in the intraocular space, thereby treating the eye.

“Contacting” the transport-resistant biological membrane or barrier with the macromolecule, biological or therapeutic agent or drug and at least a first prostanoid can be achieved in a variety of ways. In transport across the blood brain barrier, preferred methods of “contact” include administering the combination of agents systemically and administering the combination of agents directly into the carotid artery. The macromolecule, agent or drug and the prostanoid can be administered via different routes, so long as they both result in a biologically effective amount at the target membrane. In transport across the blood brain barrier, coadministration will often be preferred.

In transport into the intraocular tissues and spaces, the eye can also be “contacted” with the combination of agents by systemic administration. In certain embodiments, particularly those for ocular transport, the present invention can also be used to increase the intraocular transport or penetration of dietary components that naturally exist in the body, either with or without supplementation as part of the therapeutic regimen. The invention is thus not limited to the increased transport of exogenously administered agents, but extends to the increased transport of endogenous substances. Accordingly, increased levels of pro-vitamins, vitamins, minerals and such like can be achieved within the eye simply by provision of effective amounts of prostanoids. In such aspects of the invention, prostanoid administration will nonetheless preferably be used in combination with dietary supplementation, such as ingesting exogenous vitamins, other natural products or extracts, also including the selected intake of particular foods and food groups, such as vegetables.

To provide therapeutic agents and prostanoids to the eye, local administration will often be preferred, such as by topical or subconjunctival administration. The eye can also be contacted by the combination of agents after separate administration of the agents via different routes, including wherein one or other agent is given by systemic administration, and circulates or localizes such that an effective concentration is achieved at the eye. Thus, one agent can be added systemically, and the other locally.

Examples of suitable topical administration to the eye include administration in eye drops and by spray formulations. A further suitable topical administration route is by subconjunctival injection. The agents can also be provided to the eye periocularly or retro-orbitally. Although it is an advantage of the invention that intracameral administration is not required, this and other routes of administration are not outside the scope of the invention.

The at least a first macromolecule, biological or therapeutic agent or drug and the at least a first prostanoid may be comprised within a single composition. As such, each agent may be administered “substantially simultaneously”. However, substantially simultaneous provision is not a limitation of the single composition embodiments, as such a single composition may comprise an “instant- or readily-available formulation” of one agent and a “slow release formulation” of the other agent. Slow release formulations of ophthalmic agents are known in the art. As the prostanoid typically primes or prepares the biological membrane or barrier to facilitate increased transport or penetration, the at least a first prostanoid will preferably be the instant- or readily-available formulation, whereas the at least a first macromolecule, biological or therapeutic agent or drug will preferably be the slow release formulation.

Equally, the at least a first macromolecule, biological or therapeutic agent or drug and the at least a first prostanoid may be comprised within distinct first and second compositions. Such compositions may still be administered “substantially simultaneously”, although the distinct first and second compositions can readily be “sequentially administered”. In “sequential administration” from distinct first and second compositions, the at least a first prostanoid will preferably be administered first, and the at least a first macromolecule, biological or therapeutic agent or drug will preferably be administered at a biologically effective time after the prostanoid.

The methods of the invention therefore include providing at least a first composition comprising at least a first prostanoid to an animal or human, or to a particular tissue or site of the animal or human, at a biologically effective time prior to providing at least a second composition comprising at least one macromolecule, biological or therapeutic agent or drug.

The invention is by no means limited to the transport of a single macromolecule, biological or therapeutic agent or drug. Accordingly, at least a second, third, fourth, fifth or a plurality of macromolecules, biological or therapeutic agents or drugs may be provided in the methods, combinations, formulations, compositions and kits of the present invention.

Different combinations of two or more macromolecules, agents and drugs may be formulated within the combinations, formulations and kits of the invention such that selected macromolecules, agents and drugs are released or become biologically available at different times. In this manner, a single formulation of the invention can be used to provide a sequence or cascade of timed biological events, e.g., for use in wound healing or tissue repair. Equally, different method steps may be employed to stagger the administration of a sequence of two or more macromolecules, agents and drugs to promote an integrated biological process.

Similarly, although the effectiveness of a single prostanoid in transport is clearly established herein, the invention is not so limited. Therefore, at least a second, third, fourth, fifth or a plurality of prostanoids may be used in the methods, combinations, formulations, compositions and kits of the invention. Different combinations of prostanoids may be used with various combinations of macromolecules, agents and/or drugs, as desired.

A range of prostanoids may be used in the invention, preferably selected from the prostaglandins. Exemplary prostanoids include prostaglandin G₂, prostaglandin H₂ or an analogue or derivative thereof. Further suitable categories of prostanoids are prostaglandin A, B, D, E, F or I-series prostaglandin or analogues or derivatives thereof. Within these, PGD and PGF prostaglandins or derivatives or analogues thereof will often be preferred.

Various PGD and PGF prostaglandin derivatives and analogues are known and used in the art. Certain examples are phenyl-substituted, 3-oxa and 3-carba analogs of PGD and PGF prostaglandins. PGD₂ and PGF2α analogues are further preferred examples, with PGF2α analogues being particularly preferred. U.S. Pat. No. 6,166,073, PCT patent applications WO 00/38689 and WO 00/38690 are specifically incorporated herein by reference for purposes of further describing certain prostaglandins for use in the invention.

The term “prostaglandin”, as used herein, is not limited to naturally-occurring or “endogenous” molecules, as is sometimes used in the art, but broadly applies to synthetic, non-natural and pharmacological derivatives. This is also the meaning of “analogue” or “derivative” as used herein, i.e., a molecule with a structure based on, derived or designed from, a natural prostaglandin molecule, but including one or more modifications that do not impair the fundamental biological properties of the native prostaglandin.

There is considerable knowledge and a high level of skill in the art concerning prostaglandin analogues and derivatives, particularly pharmacological analogues and derivatives. In many cases, the pharmacological analogues and derivatives are “pro-drugs”, which are metabolized or otherwise altered in the body, whereupon they form the biologically effective molecule or molecules. Accordingly, prostanoid and prostaglandin “pro-drugs” are particularly included within the present invention.

In preferred embodiments, PGF2α analogues are contemplated for use in the invention. A “PGF2α analogue”, as used herein, is a molecule, generally a synthetic or pharmacological molecule, which substantially mimics the biological activity of PGF2α upon provision to an animal or human. “Substantially mimicking the biological activity of PGF2α” means mimicking the physiological activity, such that the same type of net effect results in vivo. Generally, this involves maintaining the important biochemical activities, and although these can be readily tested in vitro, maintaining the overall physiological activity is the key feature, irrespective of how this is achieved.

The requirement to substantially mimic the biological activity of PGF2α upon provision to an animal does not limit a PGF2α analogue to having only the properties of native PGF2α. Indeed, pharmacological PGF2α analogues typically have improved properties in one or more parameters, less side effects or such like. Therefore, a range of chemical and biological variations within the PGF2α molecule can be made to provide useful and even improved analogues for use in the present invention.

One example of a PGF2α analogue is Latanoprost (Xalatan™, Pharmacia Corporation), which is currently one of the preferred prostanoids for use in the invention. Another preferred prostanoid is the PGF2α analogue Travoprost, available as Travatan™ from Alcon.

Other preferred PGF2α analogues are commercially available and can be readily used in the invention, despite the different terminology that has been applied to such molecules. One such analogue is Unoprostone (Unoprostone Isopropylate), available as Rescula™ from Novartis, which has been termed “a docosanoid”. Another such agent is Bimatoprost, available as Lumigan™ from Allergan, which has been described as “a prostamide”, due to the presence of an amide group in place of an acetyl group.

One particular embodiment of the invention is a method of increasing the amount of a biological agent in the intraocular space of the eye, comprising contacting an eye of an animal or human with a combined effective amount of at least a first biological agent and at least a first PGF2α analogue; wherein the at least a first PGF2α analogue increases the amount of the at least a first biological agent in the intraocular space of the eye in comparison to the amount of the at least a first biological agent in the intraocular space of the eye in the absence of the at least a first PGF2α analogue.

Another particular embodiment is a method of increasing the amount of voriconazole in the intraocular space of the eye, comprising contacting an eye of an animal or human with a combined effective amount of voriconazole and at least a first prostaglandin; wherein the at least a first prostaglandin increases the amount of voriconazole in the intraocular space of the eye in comparison to the amount of voriconazole in the intraocular space of the eye in the absence of the at least a first prostaglandin.

A further particular embodiment of the invention is a method for increasing the amount of voriconazole in the intraocular space of the eye, by contacting an eye of an animal with a combined effective amount of voriconazole and Latanoprost™; wherein the Latanoprost™ increases the amount of voriconazole in the intraocular space of the eye in comparison to the amount of voriconazole in the intraocular space of the eye in the absence of the Latanoprost™. Such comparative terminology can be used to describe the methods of the invention in connection with any prostanoid and any macromolecule, agent or drug.

The range of macromolecules, agents and/or drugs for combined use with the prostanoids in the present invention is virtually limitless. Any agent that one desires to transport across a penetration- or transport-impaired biological membrane can be used, whether or not the agent typically suffers from poor transport or penetration. Naturally, the use of the invention with macromolecules, agents and/or drugs that normally exhibit poor transport or penetration is an important advance. However, the invention is widely applicable as the increased transport or penetration provided means that lower levels of the agents can be used, thus reducing costs and likely providing other benefits, such as reduced administration regimens, lower systemic or other toxicities, etc.

Examples of suitable biological agents include detectable and diagnostic agents, such as detectable dyes. Imaging is also envisioned for use in the brain and CNS.

Within the therapeutic agents for combined use with the ocular embodiments of the invention, dilating agents are one class of therapeutics, e.g., those that stimulate the radial muscles that open the pupil or those that paralyze the sphincter that closes the pupil.

Although not generally limited by transport phenomena, lubricants and artificial tear components are by no means excluded from the ophthalmic embodiments of the invention, particularly as such components can be present as part of the formulation for clinical use.

Further groups of agents for use in the ophthalmic embodiments are mydriatics, cycloplegics, miotics and cholinesterase inhibitors. Particular suitable examples include agents selected from the group consisting of physostigmine, pilocarpine, carbachol (miotics), phenylephrine, tropicamide, cyclopentolate, homatropine, scopolamine and atropine (cycloplegic).

An important group of agents for use in the invention, whether in ophthalmic or other transport embodiments, is the anti-infective class of compounds. These agents include anti-microbial, anti-bacterial, anti-viral, anti-retroviral, anti-parasitic and anti-fungal agents. A wide range of such agents is known to those of ordinary skill in the art and is approved for clinical use. Merely exemplary agents include glycopolypeptide, macrolide, beta lactam, aminoglycoside and quinolone anti-bacterial agents.

Within the anti-viral and anti-retroviral agent group, suitable examples are ganciclovir, acyclovir, famciclovir, foscarnet and cidofovir. Suitable anti-fungal agents include polypeptide anti-fungal agents. A particular example is the anti-fungal agent voriconazole. Macrolide lincosamide streptogramin B (MLS) anti-microbial agents are another currently preferred group, particularly the macrolides.

Non-limiting, but merely exemplary anti-microbial agents are those selected from the group consisting of neomycin, polymyxin B, erythromycin, trimethoprim, sulfacetamide sodium, tetracycline, oxytetracycline, norfloxacin, ciloxan, ciprofloxacin, levafloxacin, ofloxacin, gentamycin, tobramycin, vancomycin, bacitracin, cephazolin, amikacin, ketoconazole, trifluridine, caspofungin, amphotericin B and natamycin.

Other categories of useful agents are steroids, which have particular ophthalmic value. Exemplary useful steroids are those selected from the group consisting of prednisolone acetate, prednisolone phosphate, fluoromethalone, hydrocortisone, cortisone and dexamethasone.

Non-steroidal anti-inflammatory agents are also useful in the invention, and again are suitable for use in the eye. Exemplary non-steroidal antiinflammatory agents are ketorolac, indomethacin, flurbiprofen, ketoprofen, loxoprofen and diclofenac. Anti-histamines also have general and ophthalmic uses.

In the ophthalmic arena, anti-glaucoma agents are an important category of agents. Exemplary anti-glaucoma agents are those selected from the group consisting of a topical carbonic anhydrase inhibitor, a cholinesterase inhibitor, a topical beta adrenergic blocking agent (beta blocker) and a topical alpha adrenergic agonist (sympathomimetic). Particular antiglaucoma agents are exemplified by phenylephrine, acetazolamide and timolol maleate. Collagenase inhibitors are exemplified by acetyl cysteine.

Reducing agents and anti-oxidants are further examples of agents with general and ophthalmic therapeutic value, any one or more which may be used in the invention. Anti-oxidants are particularly contemplated for use in prophylaxis and therapy of macular degeneration, including age-related macular degeneration.

Nutrients, vitamins, pro-vitamins (vitamin precursors) and minerals may be used in combination with the present invention. Simply as examples, vitamin A, vitamin A analogues, vitamin C, vitamin E and zinc, and combinations thereof, are suitable for ophthalmic use. Examples of pro-vitamins are the carotenoids, such as beta-carotene, which is a carotenoid as it generates two molecules of vitamin A. Combinations of one or more of vitamins, minerals and anti-oxidants are particularly contemplated (Seddon et al., 1994; AREDS Report No. 8, 2001).

Anesthetics are another general class of agents for advantageous use with the invention. Exemplary anesthetics include those selected from the group consisting of lidocaine, marcaine, proparacaine and bupivacaine.

Anti-neoplastic (chemotherapeutic) agents are further examples, which may be used in ophthalmic or other embodiments, including for transport across the blood brain barrier, e.g., as in the treatment of brain tumors and neurological tumors and infections. Methotrexate and daunorubicin are suitable examples.

Various immune modulators, antibodies and other immune regulatory molecules may be used in the biological transport embodiments of the invention, including but not limited to the ophthalmic uses. Interferons and interleukins are examples of immune modulators. β interferon is one suitable example amongst many. Inhibitory antibodies are further suitable examples, such as antibodies against transforming growth factor beta (TGFβ2), which are useful in inhibiting conjunctival scarring (Siriwardena et al., 2002), and anti-VEGF antibodies for anti-angiogenic intervention. Antibodies that function as agonists, e.g., by receptor binding may also be used.

Where antibodies are employed, whether agonistic or antagonistic, the terms “antibody” and “immunoglobulin”, as used herein, refer broadly to any immunological binding agent, including polyclonal and monoclonal antibodies. Depending on the type of constant domain in the heavy chains, antibodies are assigned to one of five major classes: IgA, IgD, IgE, IgG, and IgM. Several of these are further divided into subclasses or isotypes, such as IgG1, IgG2, IgG3, IgG4, and the like. Generally, where antibodies rather than antigen binding regions are used in the invention, IgG and/or IgM are preferred because they are the most common antibodies in the physiological situation and because they are most easily made in a laboratory setting.

Polyclonal antibodies, obtained from antisera, may be employed in the invention. However, the use of monoclonal antibodies (MAbs) will generally be preferred. MAbs are recognized to have certain advantages, e.g., reproducibility and large-scale production, which makes them suitable for clinical treatment. The invention can thus utilize monoclonal antibodies of the murine, human, monkey, rat, hamster, rabbit and even frog or chicken origin. Due to the ease of preparation and ready availability of reagents, murine monoclonal antibodies will be used in certain embodiments and human antibodies will often be preferred.

As will be understood by those in the art, the immunological binding reagents encompassed by the term “antibody” extend to all antibodies from all species, and antigen binding fragments thereof, including dimeric, trimeric and multimeric antibodies; bispecific antibodies; chimeric antibodies; human and humanized antibodies; recombinant and engineered antibodies, and fragments thereof.

The term “antibody” is thus used to refer to any antibody-like molecule that has an antigen binding region, and this term includes antibody fragments such as Fab′, Fab, F(ab′)₂, single domain antibodies (DABs), Fv, scFv (single chain Fv), linear antibodies, diabodies, and the like. The techniques for preparing and using various antibody-based constructs and fragments are well known in the art.

In certain embodiments, the antibodies employed will be “humanized” or human antibodies. “Humanized” antibodies are generally chimeric monoclonal antibodies from mouse, rat, or other non-human species, bearing human constant and/or variable region domains (“part-human chimeric antibodies”). Mostly, humanized monoclonal antibodies for use in the present invention will be chimeric antibodies wherein at least a first antigen binding region, or complementarity determining region (CDR), of a mouse, rat or other non-human monoclonal antibody is operatively attached to, or “grafted” onto, a human antibody constant region or “framework”.

“Humanized” monoclonal antibodies for use herein may also be monoclonal antibodies from non-human species wherein one or more selected amino acids have been exchanged for amino acids more commonly observed in human antibodies. This can be readily achieved by routine recombinant technology, particularly site-specific mutagenesis. Entirely human, rather than “humanized”, antibodies may also be prepared and used in the invention. A range of techniques is available for preparing human monoclonal antibodies, including immunizing transgenic animals, such as transgenic mice, that comprise a human antibody library.

In many instances, the macromolecule, biological or therapeutic agent or drug will be proteinaceous. In these cases, the agent may be administered in the form of the protein, polypeptide or peptide itself. As well as marker proteins, the invention includes the transport or provision of transcription or elongation factors, cell cycle control proteins, kinases, phosphatases, DNA repair proteins, oncogenes, tumor suppressors, angiogenic proteins, anti-angiogenic proteins, cell surface receptors, accessory signaling molecules, transport proteins, enzymes, antigens, immunogens, apoptosis-inducing agents, anti-apoptosis agents and cytotoxins.

Particularly preferred examples include growth factors, hormones, cytokines and neurotransmitters. Vascular endothelial cell growth factor (VEGF) is one preferred example of an angiogenic growth factor, which may be used to stimulate wound healing. Anti-VEGF strategies are also contemplated for use in the invention, to inhibit angiogenesis, which can be used to treat retinopathies and other disorders.

Further suitable agents include hormone, neurotransmitter or growth factor receptors, chemokines, colony stimulating factors, chemotactic factors, extracellular matrix components, and adhesion molecules, ligands and peptides. Particular examples include growth hormone, parathyroid hormone (PTH), transforming growth factor-α (TGF-α), TGF-β1, TGF-β2, the fibroblast growth factor (FGF) family, granulocyte/macrophage colony stimulating factor (GMCSF), epidermal growth factor (EGF), platelet derived growth factor (PDGF), insulin-like growth factor (IGF), scatter factor/hepatocyte growth factor (HGF), fibrin, collagen, fibronectin, vitronectin, hyaluronic acid, RGD-containing peptides or polypeptides and angiopoietins.

Glycoproteins, such as fibronectin and vitronectin may also be used, as well as analogs or fragments thereof. Ocular tissue adhesives, as exemplified by isobutyl cyanoacrylate, a corneal mortar, as exemplified by a fibronectin/growth factor (e.g., EGF) composition, optionally with a protein crosslinking agent (e.g., aldehydes and di-imidate esters), and various admixtures of the above materials may also be used in the ocular embodiments of the invention.

A DNA, RNA, expression vector, plasmid or recombinant virus containing an expression construct can be administered, wherein the nucleic acid component will express the intended therapeutic product upon provision to cells in the target tissue.

Other suitable nucleic acid components for administration in the invention include antisense constructs and ribozymes, each of which inhibit aberrant or undesired genes or mRNA constructs, to remove harmful protein products or resultant biomolecules from target cells. Ribozymes particularly targeted to the retina are known and used in the art.

The invention is suitable for use in treating or preventing a virtually limitless range of diseases, disorders, deficiencies, conditions and infections, both within the eye and other organs and tissues of the body. Methods of treating acute or chronic infections are particularly provided, both as applied to the eye, systemically and at other locations, including the brain and central nervous system (CNS). In ophthalmic embodiments, methods of treating or preventing preseptal, orbital or periorbital cellulitis are provided.

Animals and humans to be treated by the invention include those that have, are suspected to have or are at risk for developing a microbial, bacterial, viral, retroviral, parasitic, fungal or amoebal infection. Certain examples include animals and humans that have, are suspected to have or are at risk for developing bacterial or fungal keratitis or endophthalmitis, and those that have, are suspected to have or are at risk for developing uveitis, conjunctivitis, or an intraocular or periocular inflammation. Viral infections are further exemplified by HIV-, CMV- and HSV-associated infections, including but limited to those associated with retinal disorders.

Bacterial infections to be addressed by the prophylactic and therapeutic embodiments of the invention include gram positive bacterial infections, such as staphylococcal infections, and gram negative bacterial infections, such as Pseudomonas aeruginosa infections. Fungal infections counteracted by the prophylactic and therapeutic embodiments of the invention include candidiasis and aspergillosis.

In further ophthalmic uses, the invention provides for prophylactic and/or therapeutic intervention in animals and humans that have, are suspected to have or are at risk for developing an allergy or allergies affecting the eye, diabetes, glaucoma and/or a vitamin deficiency that affects the eye. The invention further provides for prophylactic and/or therapeutic intervention in animals and humans that have, are suspected to have or are at risk for developing ocular neovascular disease, retinal and/or macular degeneration, including age-related macular degeneration.

Diseases associated with corneal neovascularization that can be treated according to the present invention include, but are not limited to, diabetic retinopathy, retinopathy of prematurity, corneal graft rejection, neovascular glaucoma and retrolental fibroplasia, epidemic keratoconjunctivitis, Vitamin A deficiency, contact lens overwear, atopic keratitis, superior limbic keratitis, pterygium keratitis sicca, sjogrens, acne rosacea, phylectenulosis, syphilis, Mycobacteria infections, lipid degeneration, chemical burns, bacterial ulcers, fungal ulcers, Herpes simplex infections, Herpes zoster infections, protozoan infections, Kaposi sarcoma, Mooren ulcer, Terrien's marginal degeneration, mariginal keratolysis, trauma, rheumatoid arthritis, systemic lupus, polyarteritis, Wegeners sarcoidosis, Scleritis, Steven's Johnson disease, periphigoid radial keratotomy, and corneal graph rejection.

Diseases associated with retinal/choroidal neovascularization that can be treated according to the present invention include, but are not limited to, diabetic retinopathy, macular degeneration, sickle cell anemia, sarcoid, syphilis, pseudoxanthoma elasticum, Pagets disease, vein occlusion, artery occlusion, carotid obstructive disease, chronic uveitis/vitritis, mycobacterial infections, Lyme's disease, systemic lupus erythematosis, retinopathy of prematurity, Eales disease, Bechets disease, infections causing a retinitis or choroiditis, presumed ocular histoplasmosis, Bests disease, myopia, optic pits, Stargarts disease, pars planitis, chronic retinal detachment, hyperviscosity syndromes, toxoplasmosis, trauma and post-laser complications.

In surgical terms, the invention further provides a range pre- and post-surgical treatments, e.g., for eye surgery, brain and neurosurgery and other procedures. For example, where an animal or human is preparing to undergo eye, brain or neurosurgery, and wherein a preoperatively combined effective amount of at least a first prostanoid and at least a first surgically beneficial agent are provided to the animal or human. One example of a “surgically beneficial agent” in eye surgery is an anesthetic.

In other surgical embodiments, the invention can be used wherein the animal or human has been subjected to eye, brain, neurosurgery or other surgical procedure, and wherein a postoperatively effective amount of at least a first prostanoid and at least a first postoperative beneficial agent are provided to the animal or human. The “postoperative beneficial agents” include anti-microbial, anti-bacterial, anti-viral, anti-retroviral, anti-parasitic and anti-fungal agents, amongst a range of other agents.

Where eye surgery is concerned, use of the invention in cataract surgery is one embodiment. Other embodiments include those connected with treatment of optic neuropathy, blunt or penetrating ocular injuries and orbital or intraocular tumors.

The present invention further provides a range of medical formulations, including ophthalmically acceptable formulations, combinations and kits. The medical formulations and medicaments of the invention generally comprise a transport effective amount of at least a first prostanoid and a biologically, diagnostically or therapeutically effective amount of at least a first biological, diagnostic or therapeutic agent. The “transport effective” and “biologically, diagnostically or therapeutically effective” amounts are generally “effective combinations”, such that a net biological, diagnostic or therapeutic effect results, notwithstanding the individual doses of the prostanoid and other agent.

Such formulations may comprises a ready release form of the at least a first prostanoid and a slow release form of the at least a first active biological agent.

Ophthalmically acceptable formulations of the invention generally comprise an ocular-transport effective amount of at least a first prostanoid and a biologically, diagnostically or therapeutically effective amount of at least a first ophthalmically active biological agent. Such formulations may also comprise a ready release form of the prostanoid and a slow release form of the ophthalmically active biological agent.

Kits of the invention generally comprise, in at least a first suitable container, a therapeutically effective combination of at least a first prostanoid and at least a first biological, diagnostic or therapeutic agent, including an ophthalmically active biological agent. The prostanoid and other agent may be comprised within a single container or within distinct containers in the kit.

At least a first apparatus for administration of the prostanoid and other agent to an animal or human may further be included, such as apparatus for administration to the eye. Examples include an eye bath, eyedropper, syringe and such like.

Such kits may further comprise instructions for using the kit in the substantially simultaneous or sequentially timed administration of the at least a first prostanoid and at least a first biological, diagnostic, therapeutic or ophthalmically active agent. The instructions may be written instructions or may be instructions in computer-readable form.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. Chemical structure of voriconazole molecule.

FIG. 2A and FIG. 2B. FIG. 2A. HPLC-UV chromatogram (detection at 255 nm) of an injection of 15 ng/μl voriconazole standard in acetonitrile. The retention time of voriconazole peak is 7.2 min. The corresponding SIR chromatogram (m/z=350) to the HPLC-UV chromatogram (detection at 255 nm) of FIG. 2A.

FIG. 3. Mass spectrum of voriconazole standard. The spectrum shows the protonated voriconazole ion ([M+H]⁺) and its acetonitrile adduct ([M+ACN]⁺).

FIG. 4A, FIG. 4B and FIG. 4C. FIG. 4A. Ion current intensity as a function of cone voltage (V) in mass detector. FIG. 4B. Ion current intensity as a function of desolvation temperature (°C) in mass detector. FIG. 4C. Ion current intensity as a function of nitrogen gas flow rate (l/h) in mass detector.

FIG. 5A and FIG. 5B. FIG. 5A. HPLC-UV chromatogram (255 nm) of blank aqueous humor. FIG. 5B. HPLC-UV chromatogram (255 nm) of blank aqueous humor spiked with 0.3 ng/μl voriconazole. Arrow indicates the voriconazole peak.

FIG. 6A and FIG. 6B. FIG. 6A. SIR chromatogram (m/z=350) of blank aqueous humor. FIG. 6B. SIR chromatogram (m/z=350) of blank aqueous humor spiked with 0.3 ng/μl voriconazole. Arrow indicates the voriconazole peak.

FIG. 7. Comparison of LC-ESI-MS with HPLC-UV using rabbit aqueous humor samples after topical application of voriconazole eye-drops. The solid circles indicate the experimental determination of voriconazole by HPLC-UV and LC-ESI-MS; the solid line is a plot of the regression equation of LC-ESI-MS on HPLC-UV (Equation: [ng Voriconazole]_{LC-ESI-MS}=0.9717×[ng Voriconazole]_{HPLC-UV}-0.0793, r²=0.9985).

FIG. 8. The average size of the ulcer [(vertical+horizontal)/2] in millimeters plotted against the number of days of treatment in the Pilot Group in Example 2.

FIG. 9. The average size of the ulcer [(vertical+horizontal)/2] in millimeters plotted against the number of days the ulcer was observed in the 5 μg Treatment Group in Example 2. Treatment was started on Day 3. Rabbit # 8 animal was excluded from the analysis because it failed to produce a corneal lesion of ≧2 mm in either dimension by day 3.

FIG. 10. The average size of the ulcer [(vertical+horizontal)/2] in millimeters plotted against the number of days the ulcer was observed in the 10 μg Treatment Group in Example 2. Treatment was started on Day 3.5.

FIG. 11. Recovery efficiency of voriconazole from solid phase extraction. Dilutions of VCZ over the range of 50 ng/ml to 1,500 ng/ml were split into two aliquots. One aliquot was analyzed without further processing by HPLC using conditions similar to those used with LC-MS, except that VCZ was measured by UV detection at 254 nm (results plotted on abscissa). The other aliquot was extracted using the SPE protocol described in Example 5, resuspended in 200 μl mobile phase, and then injected into the HPLC (results plotted on ordinate). The regression line had an r-value of 0.9989, and a slope of 0.88, indicating a recovery efficiency of VCZ of 88% with SPE.

FIG. 12A and FIG. 12B. FIG. 12A. Selected ion chromatogram of VCZ analysis by LC-MS. The ion scan from m/z=280.2±0.5 is shown for a sample of vitreous containing VCZ. The major peak for this VCZ ion fragment had a retention time of 5.27 min. FIG. 12B. Mass spectrum of ion fragments with retention time of 5.3 min in the vitreous sample shown in FIG. 12A. The protonated VCZ parent molecule has a m/z equal to 350.2. There is, however, an unrelated peak, possibly an acetonitrile adduct or cluster, that consistently occurred at 349.2. By increasing the cone voltage of the electrospray ionizer, reproducible VCZ daughter fragments were obtained at m/z=281.2 and 224.3. The 281.2 m/z peak was present in the greatest abundance.

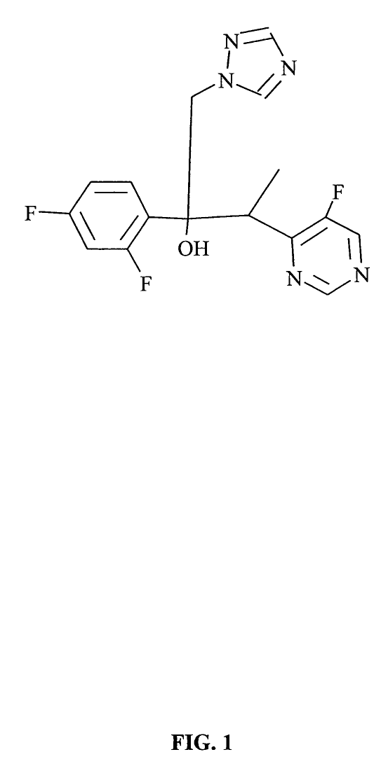

### DESCRIPTION OF ILLUSTRATIVE EMBODIMENTS

For most ophthalmic infectious diseases, particularly fungal infections, it was difficult to attain inhibitory antimicrobial concentration in ocular tissues prior to the present invention. The purpose of this invention is to solve this problem, which is achieved by providing new combinations of clinically approved agents to permit the more effective use of ocular therapeutics such as anti-microbial, anti-fungal and other agents.

The invention is first exemplified by the use of the PGF2α prostaglandin analogue, latanoprost, to enhance ocular permeability of other medications. Latanoprost is an analogue of Prostaglandin F2α, an end-product of the arachidonic acid pathway. Endogenous PGF2α is released after ocular trauma as part of the inflammatory cascade. It facilitates egress of aqueous humour from the eye via an accessory trans-scleral route under post-traumatic conditions when the normal flow of aqueous into the trabecular meshwork might be blocked by inflammatory debris or heme.

Latanoprost activates ciliary collagenase, as the free acid of the PGF2α analogue binds to FP receptors and activates the matrix metalloproteinases (collagenases) in the eye. Used at the standard dose of one drop per day, latanoprost can maintain a high steady-state level of uveoscleral aqueous outflow. Latanoprost and several other new topical prostanoids have significantly reduced the need for glaucoma filtration surgery in recent years.

The inventors hypothesized that if latanoprost could facilitate increased transudative movement of aqueous humour out of the eye by effectively increasing the size of the ocular molecular sieve, it might also enhance inward osmotic movement of pharmaceutical agents applied to the eye topically. Although a workable hypothesis in hindsight, this represents a surprising departure from thinking in the art prior to the present invention. Indeed, earlier studies have taught away from such an approach, as latanoprost has been reported not to potentiate betaxolol penetration into the eye (Osborne et al., 1999).

The present inventors first chose to evaluate a new triazole antifungal, voriconazole. The inventors' data show that voriconazole can kill amphotericin B and natamycin resistant intrastromal fungal infections with tissue concentrations in the 1-100 nanogram/ml range. They also observed similar concentrations of voriconazole in the chorioretina after 8 days' twice-daily topical treatment, suggesting the drug might be effective against fungal endophathalmitis. The present invention makes this and other therapies realizable clinically, by showing that tissue concentrations in a similar range can be attained more rapidly with a single dose of drugs such as antifungals, by pretreating eyes with prostanoids such as latanoprost.

The first studies of the inventors' indeed revealed that the penetration of voriconazole was enhanced in the eyes receiving topical preadministration of latanoprost. This established that using latanoprost to increase the permeability of the ciliary muscle and the blood-aqueous barrier allows improved and expedited penetration of pharmacologic agents into the aqueous and vitreous. In further studies in art-accepted models, paired eye results continued to show a pronounced effect of latanoprost pre-treatment on the ocular penetration of voriconazole. Thus, topical prostaglandin analogues can now be used to increase penetration of other applied drugs, either by concurrent application or pre-treatment, thereby enhancing drug penetration into ocular tissue.

Ongoing studies of the present inventors continue to show excellent penetration of drugs into the vitreous when using topically applied prostanoids and topically co-applied drugs, giving rise to many useful applications. With knowledge of the MIC values for organisms in the cornea, the penetration of drug needed to attain successful protection against, e.g., endophthalmitis in the vitreous can be calculated. The inhibitory concentrations of drug required in the cornea appear to be 3 orders of magnitude lower than those required in the plasma to control systemic infestations with the same organism. Thus, concentrations of only 1-8 nanogram per ml of drug are toxic to organisms in the eye, in contrast to the blood MIC levels of about 1-8 microgram per ml.

Such knowledge of the MIC values for organisms in the cornea highlights the important of the present invention, and shows the value of the very sensitive HPLC-MS assays described herein. The present invention is therefore able to achieve biologically and therapeutically effective enhanced drug penetration into the eye, at amounts sufficient to facilitate cidal levels of drugs and antibiotics in the vitreous, despite suggestions in the prior art that such penetration could not be achieved. Indeed, intraocular drug levels are even higher using the topical application embodiment of the invention rather than the initial subconjunctival bolus of methylcellulose/drug combination. The excellent penetration into the vitreous demonstrated in the coadministration of Example 6 is particularly important, showing that the significant clinical problem of drug penetration into the posterior chamber is effectively overcome by the present invention.

The present invention also has applicability outside the treatment of the eye, particularly in using prostanoids to “open” the blood brain barrier and thus facilitate drug penetration and/or transport into the brain and cerebrospinal fluid. The blood brain barrier is a seal of the cerebral capillary endothelial cell junction such that very few agents are able to penetrate this barrier. As the present invention shows that prostanoids function to open the barrier in the sclera of the eye, such agents are also envisioned for use in opening the blood brain barrier, e.g., following systemic administration and/or administration directly into the carotid artery.

Translating the present studies of penetration through the sclera to the CNS is based on scientifically sound principles. In fact, the eye can be viewed as an extension of the CNS, but in which the specialized tissues contain unique receptors (the photoreceptors). Many of the structural components are analogous, not only the sclera and blood brain barrier, and the aqueous humour is analogous to the cerebrospinal fluid. Moreover, several eye disorders are described as analogous to disorders of the CNS, for example, glaucoma being representative of Alzheimer's disease.

The effectiveness of therapeutic agents intended for delivery across the blood brain barrier can thus be increased following systemic administration in conjunction with prostanoids. This is exemplified by the discussion of methotrexate and interferon treatment, but is widely applicable to a range of drugs that act within the brain and CNS. It is currently envisioned that the doses of prostanoids for use in such embodiments would initially be higher than those used in ocular transport, but still within therapeutically acceptable levels without meaningful toxicities. Suitable animal models for assessing brain penetration are small animal models of neurotropic fungal infections (e.g., Aspergillus, Ramichloridium), which form abscesses that can be treated by systemically coadministered agents in accordance with the invention.

This invention therefore provides new combined uses for approved classes of drugs for use in the improved treatment of ocular diseases and disorders, and for more effective use of drugs that need to traverse the blood brain barrier. Accordingly, the formulation of the combinations, compositions and kits of the invention and the execution the prophylactic and therapeutic methods will be known to those of ordinary skill in the art in light of the present disclosure. In administering prostanoids and therapeutics to treat a range of infections and diseases, the dosages and times for administration of the agents and the therapeutic end points are known those of ordinary skill in the art. Nonetheless, additional guidance for the practice of the invention is provided in the following sections. As the present invention facilitates increased drug penetration, it will naturally be appreciated that lower doses of therapeutic agents can now be used, and that the existing therapeutic doses can be used to better effect and against a wider variety of infectious agents.

I. Anti-Infective Agents

A. Antifungals

Polypeptide antifungals are one class of agent for combined use in the invention. Caspofungin is an example of a cyclic hexapeptide, which acts to inhibit fungal cell wall synthesis of beta 1-3 D glucans, effectively rendering fungi similar to protoplasts. Caspofungin can be used to treat a variety of systemic fungal infections, including candidiasis and aspergillosis. Both of these are important causes of eye infections and, in addition, Candida species are common causes of neonatal meningitis and Aspergillus species are the most common causes of brain abscess in recipients of allogeneic bone marrow transplants. Mortality of Aspergillus brain abscesses exceeds 95%, with amphotericin B therapy and other presently available.

Caspofungin is effective against yeasts and Aspergillus at a concentration of 14 mcg/ml, and has a very slow clearance by hepatic hydroxylation, with a half life of 11-17 hours. It is given systemically once daily at 50 mg for candida and 70 mg for Aspergillus. The higher dose for Aspergillus is used because this organism is angioinvasive and tends to cause tissue infarction, in which case drug penetration is reduced. This likely accounts for the poor efficacy of antifungals in brain and eye infections caused by this organism prior to the present invention.

In the application of the invention, combined topical therapy with a prostanoid and caspofungin will preferably use caspofungin in 0.2 ml volumes, given twice and 4 times daily, at concentrations ranging from 0.1 mg/ml to 1 mg/ml. Caspofungin can be measured by HPLC, using an assay developed by Merck.

B. Antibacterials

An exemplary class of antibacterial agents for combined use in the invention is the glycopeptide class. Vancomycin is a representative member of this class. This agent is a broad spectrum drug effective against many gram positive bacteria, and acts by inhibiting cell wall synthesis. Gram positive infections (e.g., Staphylococcus aureus) are the most common causes of intraorbital infections and endophthalmitis. Vancomycin applied topically penetrates poorly into the eye, and vancomycin crosses the blood brain barrier poorly, with 10-25% penetration into cerebrospinal fluid. This is important as these organisms are common causes of bacterial meningitis. The improved ocular penetration and meningeal penetration provided by the present invention would thus be very useful in antibacterial therapy.

In the combined use of vancomycin and prostanoids as part of the invention, the prostanoid is preferably given topically with vancomycin given at 1 mg/ml and 10 mg/ml (MIC of most organisms is under 4 mcg/ml), twice daily and 4 times daily, using 0.2 ml doses. Vancomycin is readily measurable by HPLC, which effects nanogram ranges, and the assay is commercially available.

The following table, taken from Reese and Betts, 1993; Med. Let., 1992, lists antibiotics preferred for use against a given pathogenic bacterium. It is contemplated that the effectiveness of all the antibiotics listed in the following Table will be increased upon combination with the present invention.

### Antibiotics of Choice for Common Pathogens

| | | |
|---|---|---|
| Pathogen | Antibiotic of First Choice^{a} | Alternative Agents^{a} |

Gram-positive cocci

| | | |
|---|---|---|
| Staphylococcus aureus or | | |
| S. epidermidis | | |
| Non-penicillinase- | Penicillin | A first-generation cephalosporin, |
| producing | | vancomycin, imipenem, or |
| | | clindamycin; a fluoroquinolone^{b} |
| Penicillinase- | Penicillinase-resistant | A first-generation cephalosporin, |
| producing | penicillin (e.g., | vancomycin, clindamycin, imipenem, |
| | oxacillin or nafcillin) | amoxicillin-clavulanic acid, |
| | | ticarcillin-clavulanic acid, |
| | | ampicillin-sulbactam; a |
| | | fluoroquinolone^{b} |
| Methicillin-resistant | Vancomycin with or without | TMP-SMZ, minocycline |
| | gentamicin and/or | |
| | rifampin | |
| Streptococci | | |
| Group A, C, G | Penicillin | A cephalosporin^{a}, vancomycin, |
| | | erythromycin; clarithromycin; |
| | | azithromycin; clindamycin |
| Group B | Penicillin (or ampicillin) | A cephalosporin^{a}, vancomycin, or |
| | | erythromycin |
| Enterococcus | | |
| Endocarditis or | Penicillin (or ampicillin) | Vancomycin with gentamicin |
| other serious | with gentamicin | |
| infection | | |
| Uncomplicated | Ampicillin or amoxicillin | A fluoroquinolone, nitrofurantoin |
| urinary tract | | |
| infection | | |
| Viridans group | Penicillin G (with or | A cephalosporin^{a}, vancomycin |
| | without gentamicin) | |
| S. bovis | Penicillin G | A cephalosporin^{a}, vancomycin |
| S. pneumoniae | Penicillin G | A cephalosporin^{a}, erythromycin, |
| | | chloramphenicol, vancomycin |

Gram-negative cocci

| | | |
|---|---|---|
| Neisseria gonorrhoeae | Ceftriaxone | Spectinomycin, a fluoroquinolone, |
| | | cefoxitin, cefixime, cefotaxime |
| | | (see Appendix E) |
| N. meningitidis | Penicillin G | Third-generation cephalosporin, |
| | | chloramphenicol |
| Moraxella | TMP-SMZ | Amoxicillin-clavulanic acid; an |
| (Branhamella) | | erythromycin; clarithromycin |
| catarrhalis | | azithromycin, cefuroxime, cefixime, |
| | | third-generation cephalosporin, |
| | | tetracycline |

Gram-positive bacilli

| | | |
|---|---|---|
| Clostridium perfringens | Penicillin G | Chloramphenicol, metronidazole, or |
| (and Clostridium sp.) | | clindamycin |
| Listeria monocytogenes | Ampicillin with or without | TMP-SMZ |
| | gentamicin | |

Gram-negative bacilli

| | | |
|---|---|---|
| Acinetobacter | Imipenem | Tobramycin, gentamicin, or amikacin, |
| | | usually with ticarcillin or |
| | | piperacillin (or similar agent); |
| | | TMP-SMZ |
| Aeromonas hydrophila | TMP-SMZ | Gentamicin, tobramycin; imipenem; a |
| | | fluoroquinolone |
| Bacteroides | | |
| Bacteroides sp. | Penicillin G | Clindamycin, cefoxitin, |
| (oropharyngeal) | | metronidazole, chloramphenicol, |
| | | cefotetan, ampicillin-sulbactam |
| B. fragilis strains | Metronidazole | Clindamycin; ampicillin-sulbactam; |
| (gastrointestinal | | imipenem; cefoxitin^{c}; cefotetan^{c}; |
| strains) | | ticarcillin-clavulanic acid; |
| | | piperacillin^{c}; chloramphenicol; |
| | | cefmetazole^{c} |
| Campylobacter fetus, | A fluoroquinolone (adults) | A tetracycline, gentamicin |
| jejuni | or an erythromycin | |
| Enterobacter sp. | Imipenem | An aminoglycoside and piperacillin or |
| | | ticarcillin or mezlocillin; a |
| | | third-generation cephalosporin^{d}; |
| | | TMP-SMZ; aztreonam; a |
| | | fluoroquinolone |
| Escherichia coli | | |
| Uncomplicated urinary | TMP-SMZ | A cephalosporin or a fluoroquinolone |
| tract infection | | |
| Recurrent or systemic | A cephalosporin^{e} | Ampicillin with or without an |
| infection | | aminoglycoside, TMP-SMZ, oral |
| | | fluoroquinolones useful in |
| | | recurrent infections, ampicillin- |
| | | sulbactam, ticarcillin-clavulanic |
| | | acid, aztreonam |
| Haemophilus influenzae | | |
| (coccobacillary) | | |
| Life-threatening | Cefotaxime or ceftriaxone | Chloramphenicol; cefuroxime for |
| infections | | pneumonia) |
| Upper respiratory | TMP-SMZ | Ampicillin or amoxicillin; |
| infections and | | cefuroxime; a sulfonamide with or |
| bronchitis | | without an erythromycin; |
| | | cefuroxime-axetil; third-generation |
| | | cephalosporin, amoxicillin- |
| | | clavulanic acid, cefaclor, |
| | | tetracycline; clarithromycin; |
| | | azithromycin |
| Klebsiella pneumoniae | A cephalosporin^{e} | An aminoglycoside, imipenem, TMP-SMZ, |
| | | ticarcillin-clavulanic acid, |
| | | ampicillin-sulbactam, aztreonam, a |
| | | fluoroquinolone; amoxicillin- |
| | | clavulanic acid |
| Legionella spp. | Erythromycin with rifampin | TMP-SMZ; clarithromycin; |
| | | azithromycin; ciprofloxacin |
| Pasteurella multocida | Penicillin G | Tetracycline, cefuroxime, |
| | | amoxicillin-clavulanic acid, |
| | | ampicillin-sulbactam |
| Proteus sp. | Cefotaxime, ceftizoxime, or | An aminoglycoside; ticarcillin or |
| | ceftriaxone^{f} | piperacillin or mezlocillin; TMP- |
| | | SMZ; amoxicillin-clavulanic acid; |
| | | ticarcillin-clavulanic acid, |
| | | ampicillin-sulbactam; a |
| | | fluoroquinolone; aztreonam; |
| | | imipenem |
| Providencia stuartii | Cefotaxime, ceftizoxime, or | Imipenem; an aminoglycoside often |
| | ceftriaxone^{f} | combined with ticarcillin or |
| | | piperacillin or similar agent; |
| | | ticarcillin-clavulanic acid; TMP- |
| | | SMZ, a fluoroquinolone; aztreonam |

C. Quinolones

Quinolone drugs are broad spectrum against gram negative organisms. They penetrate the eye and central nervous system very poorly. Quinolones are commonly used topically for eye infections (Ciloxan, ciprofloxacin), despite poor penetration. The efficacy of quinolones can thus be improved by increasing penetration according to the present prostanoid combination invention.

The MIC for ciprofloxacin and levafloxacin range between 0.25 and 4 mcg/ml. In combined use with a prostanoid, ciprofloxacin is preferably used at the same concentration as ciloxan (0.3%) and at the typical clinical dose schedule, but after pretreatment with the prostanoid.

D. Macrolides

Macrolides have activity as antifungals and antibacterials. One macrolide of interest is the polyene amphotericin B, traditionally a compound with poor CNS and ocular penetration. Amphotericin B is poorly water soluble and penetrates the central nervous system and eye very poorly (<5% of serum concentrations, and commonly completely undetectable). Part of the reason for failure of amphotericin B (and natamycin, used for ocular infections topically) prior to the present invention is the poor tissue penetration and high systemic nephrotoxicity and infusion toxicity of these drugs. They also generate severe infusion reactions when given intravenously and cause local inflammation when given into the eye.

The MIC of most fungi for amphotericin B is <1 mcg/ml. In administering amphotericin B in combination with a prostanoid, doses of 100 mcg/ml and 1 mcg/ml twice daily are preferred. Assay for amphotericin B is accomplished by HPLC.

Gentamicin and amikacin are also representatives of the macrolide class of antimicrobials, which has broad spectrum activity against gram negative organisms such as Pseudomonas aeruginosa. These can cause endophthalmitis and meningitis, particularly following neurosurgical procedures and in the early months of life. Penetration of these agents from bloodstream to the cerebrospinal fluid, and into the eye, is less than 25% of serum concentration, when used prior to the present invention. This is important because these drugs cause nephrotoxicity and deafness at high doses, and extremely high amounts of drug cannot be given.

The MIC for gentamicin is generally less than 2 mcg/ml for most organisms targeted. Concentrations can be measured by HPLC. Amikacin is measured commercially by ELISA, and can also be measured by HPLC and bioassay.

The MIC of most organisms for amikacin is in the range of 0.5 to 4 mcg/ml. Earlier administration three times daily has been replaced recently by a high dose once daily administration systemically of 5 to 7 mg/kg intravenously. In the invention, preferably topical administration of amikacin a concentration of 1 mg/ml in 0.2 ml volumes twice daily is used with a prostanoid.

MLS antibiotics are particularly effective against gram-positive staphylococcus, streptococcus, enterococcus and bacillus, gram-negative cocci and gram-negative aerobes. Bacteria that may be attacked include Staphylococcus spp., S. sanguis, Corynebacterium diphtheria, Bacteroides spp., B. ovatus, Clostridium spp., C. difficile, B. subtilis, Lactobacillus spp., Campylobacter spp., Propionibacterium spp., Mycoplasma spp., Fusobacterium, Corynebacterium, Veillonella, S. fecalis, Nocardia farcinica, Actinobacillus actinomycetemcomitans, Group A and B streptococci, Bacillus stearothermophilus, or Pseudomonas aerugenosa.

Macrolide antibiotics include erythromycin, azithromycin, clarithromycin, roxithromycin, oleandomycin, spiramycin, josamycin, miocamycin, midecamycin, rosaramycin, troleandomycin, flurithromycin, rokitamycin or dirithromycin; lincosamide antibiotics include lincomycin, clindamycin, celesticetin; and streptogramin B antibiotics include pristinamycin and virginiamycin. Erythromycin, azithromycin, clarithromycin, lincomycin and clindamycin are often used.

E. Antivirals

Cytomegalovirus causes a frequent advancing and destructive retinal vasculitis. Herpes simplex and herpes zoster cause necrotizing retinitis, a rapidly progressing destructive process which is very difficult to control. Both processes are treated with systemically administered drugs, such as ganciclovir, acyclovir, or famciclovir. Treatment is particularly myelotoxic for ganciclovir, which is unfortunate because ganciclovir is the only agent effective against cytomegalovirus. Alternative agents, such as foscarnet and cidofovir, are complicated by severe nephrotoxicity. Accordingly, there has been developed a small pellet containing ganciclovir. This can be placed surgically in the vitreous and provides slow release drug for up to 6 months. However, a surgical procedure is required and the pellets are expensive: with a treatment running as much as $4,000 in 2002.

Accordingly, the present invention has particular advantages in the atraumatic topical delivery of ganciclovir through the sclera with the aid of a prostanoid. Preferably, ganciclovir is used at 0.01 and 0.1 mg/ml applied topically with a prostanoid. Ganciclovir can be measured by HPLC.

II. Angiogenic and Anti-Angiogenic Agents

The term “angiogenesis” refers to the generation of new blood vessels, generally into a tissue or organ. Under normal physiological conditions, humans or animals undergo angiogenesis only in very specific restricted situations. For example, angiogenesis is normally observed in wound healing, fetal and embryonic development and formation of the corpus luteum, endometrium and placenta.

A. Angiogenic Agents

Angiogenic therapies may be used in the present invention to stimulate wound healing. VEGF and FGF are primary stimulators of angiogenesis, and are particularly contemplated for use with these aspects of the invention. Other key angiogenic mediators are angiogenin and SPARC, which bind or interact with copper in their pro-angiogenic state.

B. Anti-Angiogenic Agents

Uncontrolled (persistent and/or unregulated) angiogenesis is related to various disease states, many occurring in the eye. Anti-angiogenic therapies or therapies that have, as at least part of their mode of action, an anti-angiogenic mechanism are therefore important aspects of the present invention.

The anti-angiogenic therapies may be based upon the provision of an anti-angiogenic agent or the inhibition of an angiogenic agent. Inhibition of angiogenic agents may be achieved by one or more of the methods described for inhibiting VEGF, including neutralizing antibodies, soluble receptor constructs, small molecule inhibitors, antisense, RNA aptamers and ribozymes may all be employed. For example, antibodies to angiogenin may be employed, as described in U.S. Pat. No. 5,520,914, specifically incorporated herein by reference.

As copper is both a requirement and a potent stimulus for angiogenesis, anti-copper approaches may be used, such as copper chelating agents.

In that FGF is connected with angiogenesis, FGF inhibitors may also be used. Certain examples are the compounds having N-acetylglucosamine alternating in sequence with 2-O-sulfated uronic acid as their major repeating units, including glycosaminoglycans, such as archaran sulfate. Such compounds are described in U.S. Pat. No. 6,028,061, specifically incorporated herein by reference, and may be used in combination herewith.

Numerous tyrosine kinase inhibitors useful for the treatment of angiogenesis, as manifest in various diseases states, are now known. These include, for example, the 4-aminopyrrolo[2,3-d]pyrimidines of U.S. Pat. No. 5,639,757, specifically incorporated herein by reference, which may also be used in combination with the present invention. Further examples of organic molecules capable of modulating tyrosine kinase signal transduction via the VEGFR2 receptor are the quinazoline compounds and compositions of U.S. Pat. No. 5,792,771, which is specifically incorporated herein by reference for the purpose of describing further combinations for use with the present invention in the treatment of angiogenic diseases.

Compounds of other chemical classes have also been shown to inhibit angiogenesis and may be used in combination with the present invention. For example, steroids such as the angiostatic 4,9(11)-steroids and C21-oxygenated steroids, as described in U.S. Pat. No. 5,972,922, specifically incorporated herein by reference, may be employed in combined therapy. U.S. Pat. Nos. 5,712,291 and 5,593,990, each specifically incorporated herein by reference, describe thalidomide and related compounds, precursors, analogs, metabolites and hydrolysis products, which may also be used in combination with the present invention to inhibit angiogenesis. The compounds in U.S. Pat. Nos. 5,712,291 and 5,593,990 can be administered orally.

Certain preferred components for use in inhibiting angiogenesis are angiostatin, endostatin, vasculostatin, canstatin and maspin. The protein named “angiostatin” is disclosed in U.S. Pat. Nos. 5,776,704; 5,639,725 and 5,733,876, each incorporated herein by reference. Angiostatin is a protein having a molecular weight of between about 38 kD and about 45 kD, as determined by reducing polyacrylamide gel electrophoresis, which contains approximately Kringle regions 1 through 4 of a plasminogen molecule. Angiostatin generally has an amino acid sequence substantially similar to that of a fragment of murine plasminogen beginning at amino acid number 98 of an intact murine plasminogen molecule.

The amino acid sequence of angiostatin varies slightly between species. For example, in human angiostatin, the amino acid sequence is substantially similar to the sequence of the above described murine plasminogen fragment, although an active human angiostatin sequence may start at either amino acid number 97 or 99 of an intact human plasminogen amino acid sequence. Further, human plasminogen may be used, as it has similar anti-angiogenic activity, as shown in a mouse tumor model.

Certain anti-angiogenic therapies have already been shown to cause tumor regressions, and angiostatin is one such agent. Endostatin, a 20 kDa COOH-terminal fragment of collagen XVIII, the bacterial polysaccharide CM 101, and the antibody LM609 also have angiostatic activity. However, in light of their other properties, they are referred to as anti-vascular therapies or tumor vessel toxins, as they not only inhibit angiogenesis but also initiate the destruction of tumor vessels through mostly undefined mechanisms. Their delivery according to the present invention is clearly envisioned.

Angiostatin and endostatin have become the focus of intense study, as they are the first angiogenesis inhibitors that have demonstrated the ability to not only inhibit tumor growth but also cause tumor regressions in mice. There are multiple proteases that have been shown to produce angiostatin from plasminogen including elastase, macrophage metalloelastase (MME), matrilysin (MMP-7), and 92 kDa gelatinase B/type IV collagenase (MMP-9).

MME can produce angiostatin from plasminogen in tumors and granulocyte-macrophage colony-stimulating factor (GMCSF) upregulates the expression of MME by macrophages inducing the production of angiostatin. The role of MME in angiostatin generation is supported by the finding that MME is in fact expressed in clinical samples of hepatocellular carcinomas from patients. Another protease thought to be capable of producing angiostatin is stromelysin-1 (MMP-3). MMP-3 has been shown to produce angiostatin-like fragments from plasminogen in vitro.

The mechanism of action for angiostatin is currently unclear, it is hypothesized that it binds to an unidentified cell surface receptor on endothelial cells inducing endothelial cell to undergo programmed cell death or mitotic arrest. Endostatin appears to be an even more powerful anti-angiogenesis and anti-tumor agent although its biology is less clear. Endostatin is effective at causing regressions in a number of tumor models in mice. Tumors do not develop resistance to endostatin and, after multiple cycles of treatment, tumors enter a dormant state during which they do not increase in volume. In this dormant state, the percentage of tumor cells undergoing apoptosis was increased, yielding a population that essentially stays the same size. Endostatin is thought to bind an unidentified endothelial cell surface receptor that mediates its effect. Endostatin and angiostatin are thus contemplated for delivery according to the present invention.

CM101 is a bacterial polysaccharide that has been well characterized in its ability to induce neovascular inflammation in tumors. CM101 binds to and cross-links receptors expressed on dedifferentiated endothelium that stimulates the activation of the complement system. It also initiates a cytokine-driven inflammatory response that selectively targets the tumor. It is a uniquely antipathoangiogenic agent that downregulates the expression VEGF and its receptors. CM101 is currently in clinical trials as an anti-cancer drug, and can be used in combination with this invention.

Thrombospondin (TSP-1) and platelet factor 4 (PF4) may also be used in the present invention. These are both angiogenesis inhibitors that associate with heparin and are found in platelet α-granules. TSP-1 is a large 450 kDa multi-domain glycoprotein that is constituent of the extracellular matrix. TSP-1 binds to many of the proteoglycan molecules found in the extracellular matrix including, HSPGs, fibronectin, laminin, and different types of collagen. TSP-1 inhibits endothelial cell migration and proliferation in vitro and angiogenesis in vivo. TSP-1 can also suppress the malignant phenotype and tumorigenesis of transformed endothelial cells. The tumor suppressor gene p53 has been shown to directly regulate the expression of TSP-1 such that, loss of p53 activity causes a dramatic reduction in TSP-1 production and a concomitant increase in tumor initiated angiogenesis.

PF4 is a 70aa protein that is member of the CXC ELR-family of chemokines that is able to potently inhibit endothelial cell proliferation in vitro and angiogenesis in vivo. PF4 administered intratumorally or delivered by an adenoviral vector is able to cause an inhibition of tumor growth.

Interferons and metalloproteinase inhibitors are two other classes of naturally occurring angiogenic inhibitors that can be delivered according to the present invention. The anti-endothelial activity of the interferons has been known since the early 1980s, however, the mechanism of inhibition is still unclear. It is known that they can inhibit endothelial cell migration and that they do have some anti-angiogenic activity in vivo that is possibly mediated by an ability to inhibit the production of angiogenic promoters by tumor cells. Vascular tumors in particular are sensitive to interferon, for example, proliferating hemangiomas can be successfully treated with IFNα.

Tissue inhibitors of metalloproteinases (TIMPs) are a family of naturally occurring inhibitors of matrix metalloproteases (MMPs) that can also inhibit angiogenesis and can be used in the treatment protocols of the present invention. MMPs play a key role in the angiogenic process as they degrade the matrix through which endothelial cells and fibroblasts migrate when extending or remodeling the vascular network. In fact, one member of the MMPs, MMP-2, has been shown to associate with activated endothelium through the integrin αvβ3 presumably for this purpose. If this interaction is disrupted by a fragment of MMP-2, then angiogenesis is downregulated and in tumors growth is inhibited.

There are a number of pharmacological agents that inhibit angiogenesis, any one or more of which may be used as part of the present invention. These include AGM-1470/TNP-470, thalidomide, and carboxyamidotriazole (CAI). Fumagillin was found to be a potent inhibitor of angiogenesis in 1990, and since then the synthetic analogues of fumagillin, AGM-1470 and TNP-470 have been developed. Both of these drugs inhibit endothelial cell proliferation in vitro and angiogenesis in vivo. TNP-470 has been studied extensively in human clinical trials with data suggesting that long-term administration is optimal.

Thalidomide was originally used as a sedative but was found to be a potent teratogen and was discontinued. In 1994 it was found that thalidomide is an angiogenesis inhibitor. Thalidomide is currently in clinical trials as an anti-cancer agent as well as a treatment of vascular eye diseases.

CAI is a small molecular weight synthetic inhibitor of angiogenesis that acts as a calcium channel blocker that prevents actin reorganization, endothelial cell migration and spreading on collagen IV. CAI inhibits neovascularization at physiological attainable concentrations and is well tolerated orally by cancer patients. Clinical trials with CAI have yielded disease stabilization in 49% of cancer patients having progressive disease before treatment.

Cortisone in the presence of heparin or heparin fragments was shown to inhibit tumor growth in mice by blocking endothelial cell proliferation. The mechanism involved in the additive inhibitory effect of the steroid and heparin is unclear although it is thought that the heparin may increase the uptake of the steroid by endothelial cells. The mixture has been shown to increase the dissolution of the basement membrane underneath newly formed capillaries and this is also a possible explanation for the additive angiostatic effect. Heparin-cortisol conjugates also have potent angiostatic and anti-tumor effects activity in vivo.

Further specific angiogenesis inhibitors may be used with the present invention. These include, but are not limited to, Anti-Invasive Factor, retinoic acids and paclitaxel (U.S. Pat. No. 5,716,981; incorporated herein by reference); AGM-1470 (Ingber et al., 1990; incorporated herein by reference); shark cartilage extract (U.S. Pat. No. 5,618,925; incorporated herein by reference); anionic polyamide or polyurea oligomers (U.S. Pat. No. 5,593,664; incorporated herein by reference); oxindole derivatives (U.S. Pat. No. 5,576,330; incorporated herein by reference); estradiol derivatives (U.S. Pat. No. 5,504,074; incorporated herein by reference); and thiazolopyrimidine derivatives (U.S. Pat. No. 5,599,813; incorporated herein by reference) are also contemplated for use as anti-angiogenic compositions for the combined uses of the present invention.

Compositions comprising an antagonist of an αᵥβ₃ integrin may also be used to inhibit angiogenesis as part of the present invention. As disclosed in U.S. Pat. No. 5,766,591 (incorporated herein by reference), RGD-containing polypeptides and salts thereof, including cyclic polypeptides, are suitable examples of αᵥβ₃ integrin antagonists.

The antibody LM609 against the αᵥβ₃ integrin also induces tumor regressions. Integrin αᵥβ₃ antagonists, such as LM609, induce apoptosis of angiogenic endothelial cells leaving the quiescent blood vessels unaffected. LM609 or other αᵥβ₃ antagonists may also work by inhibiting the interaction of αᵥβ₃ and MMP-2, a proteolytic enzyme thought to play an important role in migration of endothelial cells and fibroblasts.

Apoptosis of the angiogenic endothelium by LM609 may have a cascade effect on the rest of the vascular network. Inhibiting the tumor vascular network from completely responding to the tumor's signal to expand may, in fact, initiate the partial or full collapse of the network resulting in tumor cell death and loss of tumor volume. It is possible that endostatin and angiostatin function in a similar fashion. The fact that LM609 does not affect quiescent vessels but is able to cause tumor regressions suggests strongly that not all blood vessels in a tumor need to be targeted for treatment in order to obtain an anti-tumor effect.

C. VEGF Inhibition

VEGF is a multifunctional cytokine that is induced by hypoxia and oncogenic mutations. VEGF is a primary stimulant of the development and maintenance of a vascular network in embryogenesis. It functions as a potent permeability-inducing agent, an endothelial cell chemotactic agent, an endothelial survival factor, and endothelial cell proliferation factor. Its activity is required for normal embryonic development, as targeted disruption of one or both alleles of VEGF results in embryonic lethality.

VEGF is an important factor driving angiogenesis or vasculogenesis in numerous physiological and pathological processes, including wound healing, diabetic retinopathy, psoriasis and solid tumor growth.

As mentioned above, the use of one or more VEGF inhibition methods is a preferred aspect of this invention. The recognition of VEGF as a primary stimulus of angiogenesis in pathological conditions has led to various methods to block VEGF activity, any one of which may be advantageously employed herewith. Any one or more of the neutralizing anti-VEGF antibodies, soluble receptor constructs, antisense strategies, RNA aptamers and tyrosine kinase inhibitors designed to interfere with VEGF signaling may thus be used in combination with the present invention.

Suitable agents thus include neutralizing antibodies, soluble receptor constructs, tyrosine kinase inhibitors, antisense strategies, RNA aptamers and ribozymes against VEGF or VEGF receptors (Presta et al., 1997). Variants of VEGF with antagonistic properties may also be employed, as described in WO 98/16551.

Blocking antibodies against VEGF will be preferred in certain embodiments, particularly for simplicity. Monoclonal antibodies against VEGF have been shown to inhibit human tumor xenograft growth and ascites formation in mice. The antibody A4.6.1 is a high affinity anti-VEGF antibody capable of blocking VEGF binding to both VEGFR1 and VEGFR2 (Muller et al.,1998). A4.6.1 has recently been humanized by monovalent phage display techniques and is currently in Phase I clinical trials as an anti-cancer agent (Brem, 1998; Presta et al., 1997).

III. Antineoplastic Agents

There has been major concern with treatment of protected site tumors, such as glioblastomas and central nervous system lymphomas. The latter are particular common in AIDS patients. One agent commonly used, but poorly penetrating the central nervous system, is methotrexate. It is traditionally administered intrathecally for treatment of lymphomas and acute lymphocytic leukemia to reach this sequestered space.

The use of methotrexate can thus be improved by the present invention, wherein administration of methotrexate with a prostanoid is used to increase penetration of this agent into the central nervous system, as a parallel to prostanoids increasing penetration into the eye. Daunorubicin can be used in a similar manner, according to the combinations of the present invention.

Irrespective of the underlying mechanism(s), a variety of chemotherapeutic agents may be used in the combined treatment methods disclosed herein. Chemotherapeutic agents contemplated as exemplary include, e.g., tamoxifen, taxol, vincristine, vinblastine, etoposide (VP-16), adriamycin, 5-fluorouracil (5FU), camptothecin, actinomycin-D, mitomycin C, cisplatin (CDDP), combretastatin(s) and derivatives and prodrugs thereof.

As will be understood by those of ordinary skill in the art, the appropriate doses of chemotherapeutic agents will be generally around those already employed in clinical therapies wherein the chemotherapeutics are administered alone or in combination with other chemotherapeutics. By way of example only, agents such as cisplatin, and other DNA alkylating may be used. Cisplatin has been widely used to treat cancer, with efficacious doses used in clinical applications of 20 mg/m² for 5 days every three weeks for a total of three courses. Cisplatin is not absorbed orally and is therefore another good candidate for improved delivered using the present invention.

Further useful agents include compounds that interfere with DNA replication, mitosis and chromosomal segregation. Such chemotherapeutic compounds include adriamycin, also known as doxorubicin, etoposide, verapamil, podophyllotoxin, and the like. Widely used in a clinical setting for the treatment of neoplasms, these compounds are administered through bolus injections intravenously at doses ranging from 25-75 mg/m² at 21 day intervals for adriamycin, to 35-50 mg/m² for etoposide intravenously or double the intravenous dose orally.

Agents that disrupt the synthesis and fidelity of polynucleotide precursors may also be used. Particularly useful are agents that have undergone extensive testing and are readily available. As such, agents such as 5-fluorouracil (5-FU) are preferentially used by neoplastic tissue, making this agent particularly useful for targeting to neoplastic cells. Although quite toxic, 5-FU, is applicable in a wide range of carriers, including topical, however intravenous administration with doses ranging from 3 to 15 mg/kg/day being commonly used.

Exemplary chemotherapeutic agents that are useful in connection with combined therapy are listed in the table below. Each of the agents listed therein are exemplary and by no means limiting. The skilled artisan is directed to “Remington's Pharmaceutical Sciences” 15th Edition, chapter 33, in particular pages 624-652. Some variation in dosage will necessarily occur depending on the condition of the subject being treated. The physician responsible for administration will be able to determine the appropriate dose for the individual subject.

### Chemotherapeutic Agents Useful in Neoplastic Disease

| | | NONPROPRIETARY | |
|---|---|---|---|
| | | NAMES | |
| CLASS | TYPE OF AGENT | (OTHER NAMES) | DISEASE |
| Alkylating | Nitrogen Mustards | Mechlorethamine (HN₂) | Hodgkin's disease, non- |
| Agents | | | Hodgkin's lymphomas |
| | | Cyclophosphamide | Acute and chronic lymphocytic |
| | | Ifosfamide | leukemias, Hodgkin's disease, |
| | | | non-Hodgkin's lymphomas, |
| | | | multiple myeloma, |
| | | | neuroblastoma, breast, ovary, |
| | | | lung, Wilms' tumor, cervix, |
| | | | testis, soft-tissue sarcomas |
| | | Melphalan (L-sarcolysin) | Multiple myeloma, breast, ovary |
| | | Chlorambucil | Chronic lymphocytic leukemia, |
| | | | primary macroglobulinemia, |
| | | | Hodgkin's disease, non- |
| | | | Hodgkin's lymphomas |
| | Ethylenimenes and | Hexamethylmelamine | Ovary |
| | Methylmelamines | | |
| | | Thiotepa | Bladder, breast, ovary |
| | Alkyl Sulfonates | Busulfan | Chronic granulocytic leukemia |
| | Nitrosoureas | Carmustine (BCNU) | Hodgkin's disease, non- |
| | | | Hodgkin's lymphomas, primary |
| | | | brain tumors, multiple myeloma, |
| | | | malignant melanoma |
| | | Lomustine (CCNU) | Hodgkin's disease, non- |
| | | | Hodgkin's lymphomas, primary |
| | | | brain tumors, small-cell lung |
| | | Semustine | Primary brain tumors, stomach, |
| | | (methyl-CCNU) | colon |
| | | Streptozocin | Malignant pancreatic |
| | | (streptozotocin) | insulinoma, malignant carcinoid |
| | Triazines | Dacarbazine (DTIC; | Malignant melanoma, Hodgkin's |
| | | dimethyltriazenoimida | disease, soft-tissue sarcomas |
| | | zolecarboxamide) | |
| Anti- | Folic Acid Analogs | Methotrexate | Acute lymphocytic leukemia, |
| metabolites | | (amethopterin) | choriocarcinoma, mycosis |
| | | | fungoides, breast, head and |
| | | | neck, lung, osteogenic sarcoma |
| | Pyrimidine Analogs | Fluouracil | Breast, colon, stomach, |
| | | (5-fluorouracil; 5-FU) | pancreas, ovary, head and neck, |
| | | Floxuridine (fluorodeoxyuridine; | urinary bladder, premalignant |
| | | FUdR) | skin lesions (topical) |
| | | Cytarabine (cytosine | Acute granulocytic and acute |
| | | arabinoside) | lymphocytic leukemias |
| | Purine Analogs and | Mercaptopurine | Acute lymphocytic, acute |
| | Related Inhibitors | (6-mercaptopurine; | granulocytic and chronic |
| | | 6-MP) | granulocytic leukemias |
| | | Thioguanine | Acute granulocytic, acute |
| | | (6-thioguanine; TG) | lymphocytic and chronic |
| | | | granulocytic leukemias |
| | | Pentostatin | Hairy cell leukemia, mycosis |
| | | (2-deoxycoformycin) | fungoides, chronic lymphocytic |
| | | | leukemia |
| Natural | Vinca Alkaloids | Vinblastine (VLB) | Hodgkin's disease, non- |
| Products | | | Hodgkin's lymphomas, breast, |
| | | | testis |
| | | Vincristine | Acute lymphocytic leukemia, |
| | | | neuroblastoma, Wilms' tumor, |
| | | | rhabdomyosarcoma, Hodgkin's |
| | | | disease, non-Hodgkin's |
| | | | lymphomas, small-cell lung |
| | Epipodophyllotoxins | Etoposide | Testis, small-cell lung and other |
| | | Tertiposide | lung, breast, Hodgkin's disease, |
| | | | non-Hodgkin's lymphomas, |
| | | | acute granulocytic leukemia, |
| | | | Kaposi's sarcoma |
| | Antibiotics | Dactinomycin | Choriocarcinoma, Wilms' tumor, |
| | Antibiotics, continued | (actinomycin D) | rhabdomyosarcoma, testis, |
| | | | Kaposi's sarcoma |
| | | Daunorubicin | Acute granulocytic and acute |
| | | (daunomycin; | lymphocytic leukemias |
| | | rubidomycin) | |
| | | Doxorubicin | Soft-tissue, osteogenic and |
| | | | other sarcomas; Hodgkin's |
| | | | disease, non-Hodgkin's |
| | | | lymphomas, acute leukemias, |
| | | | breast, genitourinary, thyroid, |
| | | | lung, stomach, neuroblastoma |
| | | Bleomycin | Testis, head and neck, skin, |
| | | | esophagus, lung and |
| | | | genitourinary tract; Hodgkin's |
| | | | disease, non-Hodgkin's |
| | | | lymphomas |
| | | Plicamycin | Testis, malignant hypercalcemia |
| | | (mithramycin) | |
| | | Mitomycin | Stomach, cervix, colon, breast, |
| | | (mitomycin C) | pancreas, bladder, head and |
| | | | neck |
| | Enzymes | L-Asparaginase | Acute lymphocytic leukemia |
| | Biological Response | Interferon alfa | Hairy cell leukemia, Kaposi's |
| | Modifiers | | sarcoma, melanoma, carcinoid, |
| | | | renal cell, ovary, bladder, non- |
| | | | Hodgkin's lymphomas, mycosis |
| | | | fungoides, multiple myeloma, |
| | | | chronic granulocytic leukemia |
| Miscellaneous | Platinum | Cisplatin (cis-DDP) | Testis, ovary, bladder, head and |
| Agents | Coordination | Carboplatin | neck, lung, thyroid, cervix, |
| | Complexes | | endometrium, neuroblastoma, |
| | | | osteogenic sarcoma |
| | Anthracenedione | Mitoxantrone | Acute granulocytic leukemia, |
| | | | breast |
| | Substituted Urea | Hydroxyurea | Chronic granulocytic leukemia, |
| | | | polycythemia vera, essental |
| | | | thrombocytosis, malignant |
| | | | melanoma |
| | Methyl Hydrazine | Procarbazine | Hodgkin's disease |
| | Derivative | (N-methylhydrazine, | |
| | | MIH) | |
| | Adrenocortical | Mitotane (o,p′-DDD) | Adrenal cortex |
| | Suppressant | Aminoglutethimide | Breast |
| Hormones | Adreno- | Prednisone (several | Acute and chronic lymphocytic |
| and | corticosteroids | other equivalent | leukemias, non-Hodgkin's |
| Antagonists | | preparations | lymphomas, Hodgkin's disease, |
| | | available) | breast |
| | Progestins | Hydroxyprogesterone | Endometrium, breast |
| | | caproate | |
| | | Medroxyprogesterone | |
| | | acetate | |
| | | Megestrol acetate | |
| | Estrogens | Diethylstilbestrol | Breast, prostate |
| | | Ethinyl estradiol (other | |
| | | preparations | |
| | | available) | |
| | Antiestrogen | Tamoxifen | Breast |
| | Androgens | Testosterone propionate | Breast |
| | | Fluoxymesterone (other | |
| | | preparations | |
| | | available) | |
| | Antiandrogen | Flutamide | Prostate |
| | Gonadotropin- | Leuprolide | Prostate |
| | releasing hormone | | |
| | analog | | |

Anti-tubulin drugs are drugs that exert their effects via interfering with tubulin activity. As tubulin functions are essential to mitosis and cell viability, certain “anti-tubulin drugs” are powerful chemotherapeutic agents. Some of the more well known and currently preferred anti-tubulin drugs for use in combination with these aspects of the invention are colchicine; taxanes, such as taxol; vinca alkaloids, such as vinblastine, vincristine and vindescine; and combretastatins. Other suitable anti-tubulin drugs are cytochalasins (including B, J, E), dolastatin, auristatin PE, paclitaxel, ustiloxin D, rhizoxin, 1069C85, colcemid, albendazole, azatoxin and nocodazole.

As described in U.S. Pat. Nos. 5,892,069, 5,504,074 and 5,661,143, each specifically incorporated herein by reference, combretastatins are estradiol derivatives that generally inhibit cell mitosis. Exemplary combretastatins that may be used in conjunction with the invention include those based upon combretastatin A, B and/or D and those described in U.S. Pat. Nos. 5,892,069, 5,504,074 and 5,661,143. Combretastatins A-1, A-2, A-3, A-4, A-5, A-6, B-1, B-2, B-3 and B-4 are exemplary of the foregoing types.

U.S. Pat. No. 5,569,786 and 5,409,953, are incorporated herein by reference for purposes of describing the isolation, structural characterization and synthesis of each of combretastatin A-1, A2, A-3, B-1, B-2, B-3 and B-4 and formulations and methods of using such combretastatins to treat neoplastic growth. Any one or more of such combretastatins may be used in conjunction with the present invention.

Combretastatin A-4, as described in U.S. Pat. Nos. 5,892,069, 5,504,074, 5,661,143 and 4,996,237, each specifically incorporated herein by reference, may also be used herewith. U.S. Pat. No. 5,561,122 is further incorporated herein by reference for describing suitable combretastatin A-4 prodrugs, which are contemplated for combined use with the present invention.

U.S. Pat. No. 4,940,726, specifically incorporated herein by reference, particularly describes macrocyclic lactones denominated combretastatin D-1 and Combretastatin D-2, each of which may be used in combination with the compositions and methods of the present invention. U.S. Pat. No. 5,430,062, specifically incorporated herein by reference, concerns stilbene derivatives and combretastatin analogues with anti-cancer activity that may be used in combination with the present invention.

IV. Additional Therapeutics

A. Immune Modulators

Another category of agents for combined use with the invention is immune modulators, including protein modulators such as cytokines. Within this group, beta interferon is one preferred agent, as interferon currently penetrates the central nervous system poorly (as do most peptides/proteins). Interferon beta is used now for treatment of multiple sclerosis, and can be rendered more effective by increased penetration of the blood brain barrier achieved by this invention.

Interferon beta is typically used at 30 mcg intramuscularly on a weekly basis, which would be supplemented by prostanoid administration according to the invention. Measurement of interferon is typically performed by ELISA. Assays of this and other drugs in the cerebrospinal fluid are performed to demonstrate increased penetration across the blood brain barrier.

Cytokine therapy also has proven to be effective, both alone and as an effective partner for combined therapeutic regimens. Various cytokines may be employed in such combined approaches. Examples of cytokines include IL-1α IL-1β, IL-2, IL-3, IL-4, IL-5, IL-6, IL-7, IL-8, IL-9, IL-10, IL-11, IL-12, IL-13, TGF-β, GM-CSF, M-CSF, G-CSF, TNFα, TNFβ, LAF, TCGF, BCGF, TRF, BAF, BDG, MP, LIF, OSM, TMF, PDGF, IFN-α, IFN-β, IFN-γ. Cytokines are administered according to standard regimens, consistent with clinical indications such as the condition of the patient and relative toxicity of the cytokine. Uteroglobins may also be used to prevent or inhibit metastases (U.S. Pat. No. 5,696,092; incorporated herein by reference).

B. Ocular Therapeutics

Although there is considerable functional overlap, and the foregoing anti-infective, angiogenic, anti-angiogenic and antineoplastic agents and immune modulators may all be used in ocular embodiments, the present invention particularly contemplates the improved administration of agents optimized for ocular treatment.

A broad range of ocular therapeutics may be used, such as beta-carotene, histidine, CNTF, GNDF, TGFβ, palmitate, melatonin antagonists, melatonin agonists, dopamine antagonists, dopamine agonists, dopamine hydrochloride, 13-cis-retinoic acid and 13-cis-retinoic acid derivatives and various combinations thereof.

Melatonin antagonists such as those described in U.S. Pat. Nos. 5,314,908, 5,283,343, 5,151,446 and 4,880,826, each specifically incorporated herein by reference, are also contemplated for use in combined aspects of the present invention. Melatonin agonists such as those described in U.S. Pat. No. 5,151,446 are further contemplated for use in combined aspects of the present invention.

The use of the dopamine antagonists, as well as 4-piperidino-4′-fluoro-butyrophenones, especially haloperidol, trifluperidol, and moperone, clofluperol, pipamperone, lemperone, droperidol and loxapine, are contemplated for use in certain combined aspects of the present invention. Dopamine agonists contemplated for combined use include, but are not limited to, 5′a-2-bromo-12′-hyseozy-2′-(1-methylethyl)-5′-(2-methylpropyl)-ergotaman-3′,6′, 18-trione (bromocriptine mesylate), N-(methyl-4-(2-cyanophenyl)-piperazinyl-3-methylbenzamide) (PD 168077 maleate), (4a-R-trans)-4,4a,5,6,7,8,8a,9-octahydro-5-propyl-1H-pyrazolo-[3,4-g]-quinoline (quinperole; (−) quinperole dihydrochloride) and 2-(4-(1,3-benzodioxol-5-yl-methyl)-1-piperazinyl)-pyrimidine (peribedil hydrochloride).

V. Pharmaceutical Compositions

Pharmaceutical compositions of the present invention will generally comprise an effective amount of at least a first prostanoid or prostaglandin and at least a first biological agent for transport into the eye, such as an ophthalmically active diagnostic or therapeutic agent. The pharmaceutical compositions will generally be dissolved or dispersed in at least a first pharmaceutically acceptable carrier or aqueous medium. The at least a first prostanoid or prostaglandin and the at least a first biological agent for transport into the eye, i.e., the ophthalmically active diagnostic or therapeutic agent, may be combined within a single pharmaceutical composition or maintained within two or more distinct pharmaceutical compositions. The following descriptions are applicable to the pharmaceutical compositions of the invention, irrespective of whether the agents are formulated in single composition or multiple compositions.

The phrases “pharmaceutically or pharmacologically acceptable” refer to molecular entities and compositions that do not produce an adverse, allergic or other untoward reaction when administered to an animal, or a human, as appropriate. As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

A. Injectable Formulations

The agents of the present invention will often be formulated for injection, particularly by subconjunctival injection. The preparation of an aqueous composition that contains one or more prostanoids or prostaglandins, and optionally other biological and ophthalmically active agents, will be known to those of skill in the art in light of the present disclosure. Typically, such compositions can be prepared as injectables, either as liquid solutions or suspensions; solid forms suitable for using to prepare solutions or suspensions upon the addition of a liquid prior to injection can also be prepared; and the preparations can also be emulsified. Although not necessarily preferred, the agents of the present invention can also be formulated for parenteral administration, e.g., formulated for injection via the intravenous, intramuscular, subcutaneous or other such routes.

Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi.

Compositions comprising the agents of the present invention can be formulated into a composition in a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier can also be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Even more prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Upon formulation, solutions will be administered in a manner compatible with the dosage formulation and in such amount as is therapeutically effective. Formulations are easily administered in a variety of dosage forms, such as the type of injectable solutions described above. Suitable pharmaceutical compositions in accordance with the invention will generally include an amount of one or more of the agents of the present invention admixed with an acceptable pharmaceutical diluent or excipient, such as a sterile aqueous solution, to give a range of final concentrations, depending on the intended use. The techniques of preparation are generally well known in the art as exemplified by Remington's Pharmaceutical Sciences, 16th Ed. Mack Publishing Company, 1980, incorporated herein by reference. Moreover, for human administration, preparations should meet sterility, pyrogenicity, general safety and purity standards as required by FDA Office of Biological Standards.

The therapeutically effective doses are readily determinable using animal models, as shown in the studies detailed herein, and preferably, according to the clinical doses already used in other embodiments. Experimental animals are frequently used to optimize appropriate therapeutic doses prior to translating to a clinical environment. Such models are known to be very reliable in predicting effective clinical strategies in this field. The inventors have used such art-accepted rodent models to determine working ranges of agents that provide beneficial transport effects.

It is contemplated that certain benefits may result from the manipulation of the agents of the present invention to provide them with a longer in vivo half-life. Slow release formulations are generally designed to give a constant drug level over an extended period. Increasing the half-life of a drug is intended to result in high intraocular levels upon administration, which levels are maintained for a longer time, but which levels generally decay depending on the pharmacokinetics of the construct.

B. Ophthalmic Solutions

In addition to the compounds formulated for injection, topical ophthalmic formulations are particularly appropriate for many of the conditions described herein. Methods for the determination of preferred and optimal dosages for various conditions will be evident to those of skill in the art in light of the dosages used in the art for other clinical indications and in light of the data and teaching in the instant specification.

The prostanoids or prostaglandins, and optionally other biological and ophthalmically active agents, of the present invention may thus be advantageously used for the preparation of pharmaceutical compositions suitable for use as topical ophthalmic solutions. Such ophthalmic preparations may be prepared in accordance with conventional pharmaceutical practice, see for example “Remington's Pharmaceutical Sciences” 15th Edition, pages 1488 to 1501 (Mack Publishing Co., Easton, Pa.).

The ophthalmic preparations will contain one or more prostanoids or prostaglandins, and optionally other biological and ophthalmically active agents, in any suitable concentration, such as from about 0.01 to about 1% by weight, preferably from about 0.05 to about 0.5% in a pharmaceutically acceptable solution, suspension or ointment. Some variation in concentration will necessarily occur, depending on the particular compound employed, the condition of the subject to be treated and the like, and the person responsible for treatment will determine the most suitable concentration for the individual subject. The ophthalmic preparation will preferably be in the form of a sterile aqueous solution containing, if desired, additional ingredients, for example preservatives, buffers, tonicity agents, antioxidants and stabilizers, nonionic wetting or clarifying agents, viscosity-increasing agents and the like.

Suitable preservatives for use in such a solution include benzalkonium chloride, benzethonium chloride, chlorobutanol, thimerosal and the like. Suitable buffers include boric acid, sodium and potassium bicarbonate, sodium and potassium borates, sodium and potassium carbonate, sodium acetate, sodium biphosphate and the like, in amounts sufficient to maintain the pH at between about pH 6 and pH 8, and preferably, between about pH 7 and pH 7.5. Suitable tonicity agents are dextran 40, dextran 70, dextrose, glycerin, potassium chloride, propylene glycol, sodium chloride, and the like, such that the sodium chloride equivalent of the ophthalmic solution is in the range 0.9 plus or minus 0.2%.

Suitable antioxidants and stabilizers include sodium bisulfite, sodium metabisulfite, sodium thiosulfite, thiourea and the like. Suitable wetting and clarifying agents include polysorbate 80, polysorbate 20, poloxamer 282 and tyloxapol. Suitable viscosity-increasing agents include dextran 40, dextran 70, gelatin, glycerin, hydroxyethylcellulose, hydroxmethylpropylcellulose, lanolin, methylcellulose, petrolatum, polyethylene glycol, polyvinyl alcohol, polyvinylpyrrolidone, carboxymethylcellulose and the like. The ophthalmic preparation will be administered topically to the eye of the subject in need of treatment by conventional methods, for example in the form of drops or by bathing the eye in the ophthalmic solution.

C. Sustained Release Formulations

Formulations are easily administered in a variety of dosage forms, such as the type of injectable solutions described above, but other pharmaceutically acceptable forms are also contemplated, including pharmaceutical “slow release” compositions. Slow release formulations are generally designed to give a constant drug level over an extended period and may be used to deliver the same or different agents in accordance with the present invention.

Pharmaceutical “slow release” capsules or “sustained release” compositions or preparations may also be used. Slow release formulations are generally designed to give a constant drug level over an extended period. The slow release formulations are typically implanted in the vicinity of the disease site, and can be implanted in the eye.

Suitable examples of sustained-release preparations include semipermeable matrices of solid hydrophobic polymers, which matrices are in the form of shaped articles, e.g., films or microcapsule. Examples of sustained-release matrices include polyesters; hydrogels, for example, poly(2-hydroxyethyl-methacrylate) or poly(vinylalcohol); polylactides, e.g., U.S. Pat. No. 3,773,919; copolymers of L-glutamic acid and γ ethyl-L-glutamate; non-degradable ethylene-vinyl acetate; degradable lactic acid-glycolic acid copolymers, such as the Lupron Depot™ (injectable microspheres composed of lactic acid-glycolic acid copolymer and leuprolide acetate); and poly-D-(−)-3-hydroxybutyric acid.

While polymers such as ethylene-vinyl acetate and lactic acid-glycolic acid enable release of molecules for over 100 days, certain hydrogels release proteins for shorter time periods. When encapsulated proteins remain in the body for a long time, they may denature or aggregate as a result of exposure to moisture at 37° C., thus reducing biological activity and/or changing immunogenicity. Rational strategies are available for stabilization depending on the mechanism involved. For example, if the aggregation mechanism involves intermolecular S—S bond formation through thio-disulfide interchange, stabilization is achieved by modifying sulfhydryl residues, lyophilizing from acidic solutions, controlling moisture content, using appropriate additives, developing specific polymer matrix compositions, and the like.

IV. Therapeutic Kits

The present invention also provides therapeutic kits comprising the agents of the present invention described herein. Such kits will generally contain, in suitable container, a pharmaceutically acceptable formulation of at least a first prostanoid or prostaglandin and another biological or ophthalmically active agent, in accordance with the overall invention. The kits may contain other pharmaceutically acceptable formulations, including a variety of ophthalmically beneficial drugs.

The kits may have a single container that contains the prostanoid agent and additional component(s), or they may have distinct containers for each desired agent. Certain preferred kits of the present invention include at least a first prostanoid or prostaglandin packaged in a kit for use in combination with the co-administration of a second therapeutic agent, such as an anti-fungal agent. In such kits, the components may be pre-complexed, either in a molar equivalent combination, or with one component in excess of the other; or each of the components of the kit may be maintained separately within distinct containers prior to administration to a patient.

When the components of the kit are provided in one or more liquid solutions, the liquid solution is an aqueous solution, with a sterile aqueous solution being particularly preferred. However, the components of the kit may be provided as dried powder(s). When reagents or components are provided as a dry powder, the powder can be reconstituted by the addition of a suitable solvent. It is envisioned that the solvent may also be provided in another container.

The container means of the kit will generally include at least one vial, test tube, flask, bottle, syringe or other container means, into which the prostanoids or prostaglandins, and other biological and ophthalmically active agents, may be placed and, preferably, suitably aliquoted. Where additional components are included, the kit will also generally contain a second vial or other container into which these are placed, enabling the administration of separated designed doses. The kits may also comprise a second/third container means for containing a sterile, pharmaceutically acceptable buffer or other diluent.

The kits may also contain a means by which to administer the prostanoids or prostaglandins and biological or ophthalmically active agents to an animal or patient, e.g., one or more needles or syringes, or an eye dropper, pipette, or other such like apparatus, from which the formulation may be injected into the animal or applied to the eye or eyes or a diseased area of the eye or eyes. The kits of the present invention will also typically include a means for containing the vials, or such like, and other component, in close confinement for commercial sale, such as, e.g., injection or blow-molded plastic containers into which the desired vials and other apparatus are placed and retained.

The following examples are included to demonstrate preferred embodiments of the invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLE 1

### Analysis of Aqueous Humor by Liquid Chromatography-Electrospray Ionization Mass Spectrometry

The present example describes the development of methods particularly suited to the analysis of therapeutic agents in aqueous humor based on liquid chromatography-electrospray ionization mass spectrometry (LC-ESI-MS). The methods are exemplified by the analysis of voriconazole in aqueous humor.

The separation was achieved on a reversed-phase C18 column eluted by 70% acetonitrile-30% water-0.01% TFA. The correlation between the concentration of voriconazole to peak area was linear (r²=0.9989) between 0.04 ng to 10 ng, with a coefficient of variance of less than 3%. The detection limit was estimated to be 0.1 ng/ml voriconazole in aqueous humor, 500 times more sensitive than the conventional HPLC-UV detection method. Both intra-day and inter-day imprecision were less than 3% over the whole analytical range. Parallel analyses of voriconazole samples by LC-ESI-MS and by HPLC-UV showed that the two methods were highly correlated (r²=0.9985).

LC-ESI-MS was used to the determine voriconazole levels achieved in the aqueous humor of the rabbit eye, following topical application of 5 μg or 10 μg voriconazole in the form of eyedrops for eleven days b.i.d. The lower dosage produced an aqueous humor concentration of 7.34±5.88 ng/ml, while the higher dosage produced a concentration of 14.7±12.99 ng/ml.

A. Materials and Methods

Voriconazole (UK-109,496) was obtained from Pfizer Central Research (Sandwich, UK). The activity of this agent as supplied was 99.9%. For use in the animal studies, it was dissolved in Noble Agar at a concentration of 50 μg/ml or 100 μg/ml. HPLC grade acetonitrile was purchased from Fisher Scientific (USA). Trifluoroacetic acid (TFA) was purchased from Sigma (USA). The water used in the mobile phase was of Milli-Q grade (Millipore, Mass., USA).

1. HPLC Conditions

The HPLC system consisted of a Waters 2690 solvent delivery system including an auto-sampler and photodiode array detector. The Delta PAK C₁₈ analytical column, (15-μm pore, 300×3.9 mm, provided by Waters Associates) was eluted by an acetonitrile:water:TFA mixture in the ratio 70:29.99:0.01 by volume. The flow rate was 0.5 ml/min.

2. ESI-MS Conditions

The ESI-MS system used in this study was the Micromass Platform LCZ (UK), coupled to the HPLC system. The optimized settings in the MS detector were as follows. The nitrogen gas flow was maintained at 350 l/h. The capillary and cone voltages were set to 3.5V and 10V, respectively. The source temperature and desolvation temperature were set to 140° C. and 425° C., respectively. All mass spectra were recorded under a full scan operation for positive ions, with a scan range from m/z 50 to 600. The quantification was carried out with the selected-ion recording (SIR) mode by monitoring the protonated molecular ion (m/z=350).

3. Calibration Curve

The determination of voriconazole was based on the external standard method. For the preparation of calibration standards, a known amount of pure voriconazole was added to blank aqueous humor to obtain voriconazole concentrations of 0.02 ng/μl˜2.5 ng/μl. Six-point calibration curves (triplicate injections) were created for the range from 0.04 ng to 10 ng by plotting the peak area of protonated voriconazole molecular ion (m/z 350) against the amount of voriconazole injected into the column.

4. Aqueous Humor Specimens and Sample Preparation

To study the penetration of voriconazole into the eye, albino rabbits each weighing ca. 2-kg were treated with topical eye drops of voriconazole at a low dose (50 μg/ml) or at a high dose (100 μg/ml) twice a day for eleven days. In each session, the drug was delivered in a volume of 0.1 ml to the eye, so that in the low-dose regimen, the animal received 5 μg per treatment, and in the high-dose regimen the animal received 10 μg per treatment. Thus, the animals in the low-dose group (n=4) received a total cumulative dose of 0.11 mg, and the high-dose animals (n=7) received a total cumulative dose of 0.22 mg. Samples of blank aqueous humor were obtained from 5 eyes of untreated rabbits.

Aqueous humor samples were obtained from the anterior chamber of the eye by entering the limbus with a 30-ga needle fitted to a 1-ml syringe. The samples were obtained between 15 and 60 min following the last voriconazole treatment. The aqueous humor is a transparent liquid that fills the anterior chamber between the cornea and lens. It contains very little protein. Therefore, the aqueous humor samples required no further preparation, and 2 μl aliquots were directly injected into the column for analysis.

5. Recovery, Precision and Accuracy

The recovery was determined by comparing the peak area of a blank aqueous humor sample premixed with a known amount of voriconazole with a sample containing the same concentration in pure methanol. For intra-assay precision, aqueous humor samples spiked with voriconazole at three different concentrations (75 μg/l, 300 μg/l and 750 μg/l) were analyzed. For the inter-assay precision, the above samples were analyzed on 3 subsequent days. Accuracy was measured using aqueous humor spiked with voriconazole at five different concentrations (60 μg/l, 200 μg/500 μg/l, 800 μg/l and 1000 μg/l), and calculated as the deviation from the theoretical values.

B. Results and Discussion

The mobile phase used in this study was modified from the one described by Gage and Stopher (1998). In order to avoid using a non-evaporating buffer system, such as phosphate salt, in the LC-MS system, the inventors used a mobile phase consisting of 70% acetonitrile/30% water/0.01% TFA.

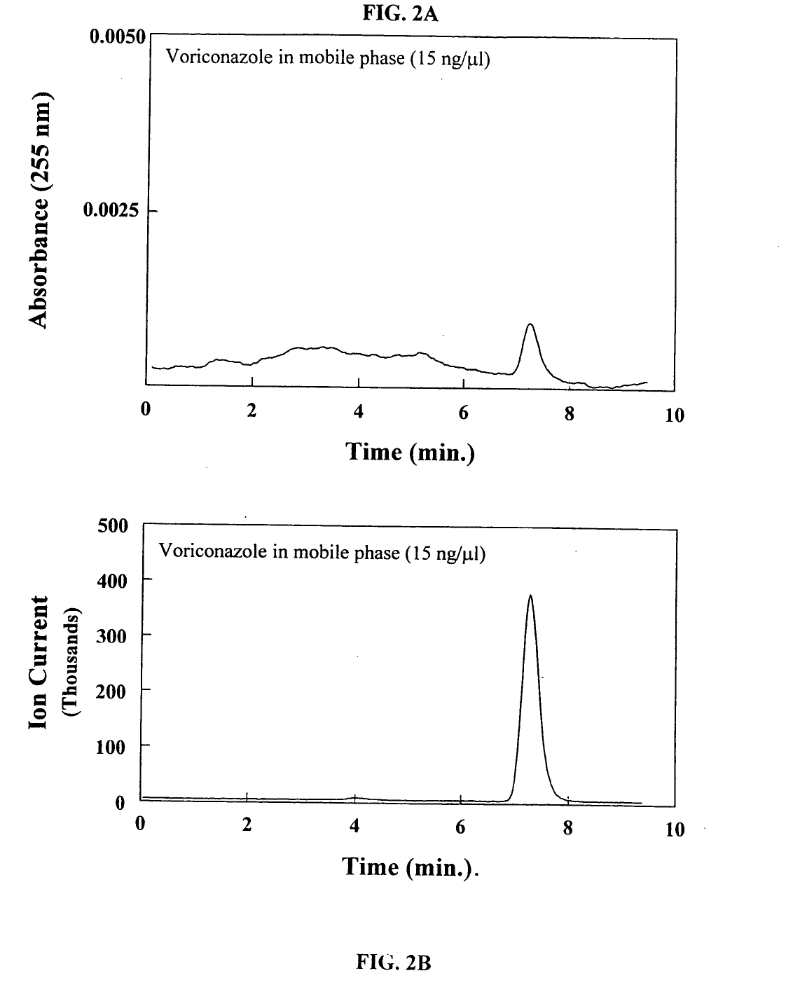

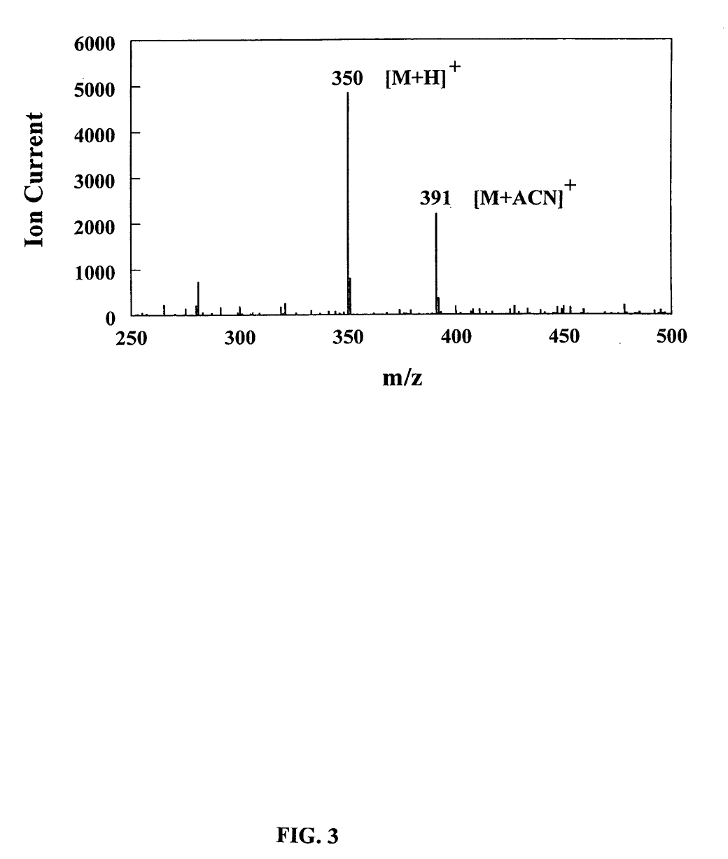

With an injection of 15 ng of pure voriconazole, the typical HPLC-UV chromatogram at a detection wavelength of 255 nm is shown in FIG. 2A, while the SIR chromatogram of the same sample is shown in FIG. 2B. The retention time (RT) for the voriconazole peak is 7.2 min. The corresponding mass spectrum is shown in FIG. 3. Both the protonated ion ([M+H]⁺, m/z=350) and its acetonitrile adduct ([M+ACN]⁺, m/z=391) were observed in the mass spectrum. Quantification of voriconazole was based only on the dominant mass peak ([M+H]⁺, m/z=350).

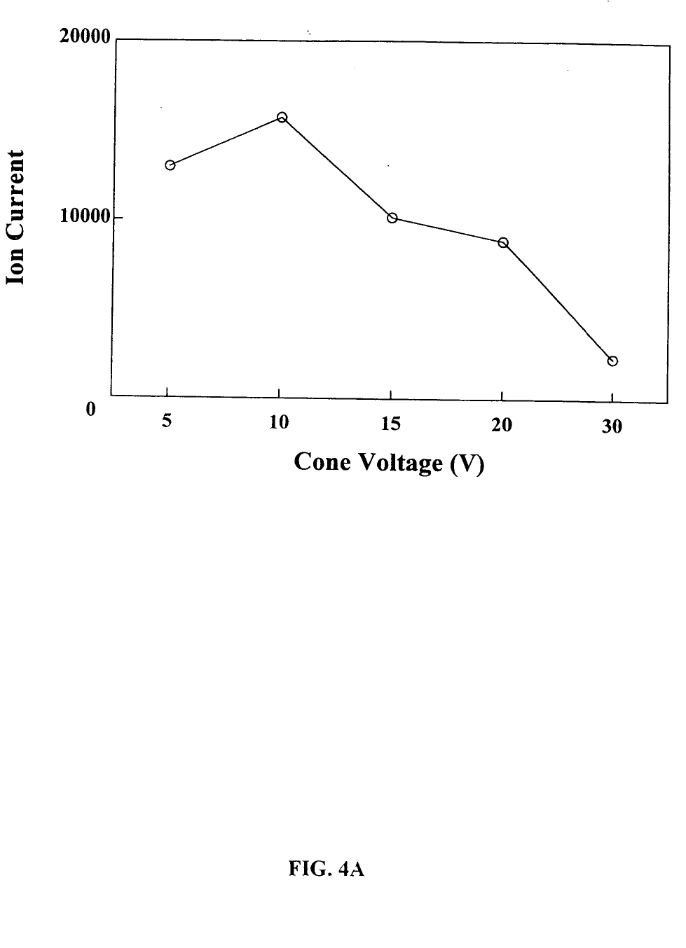

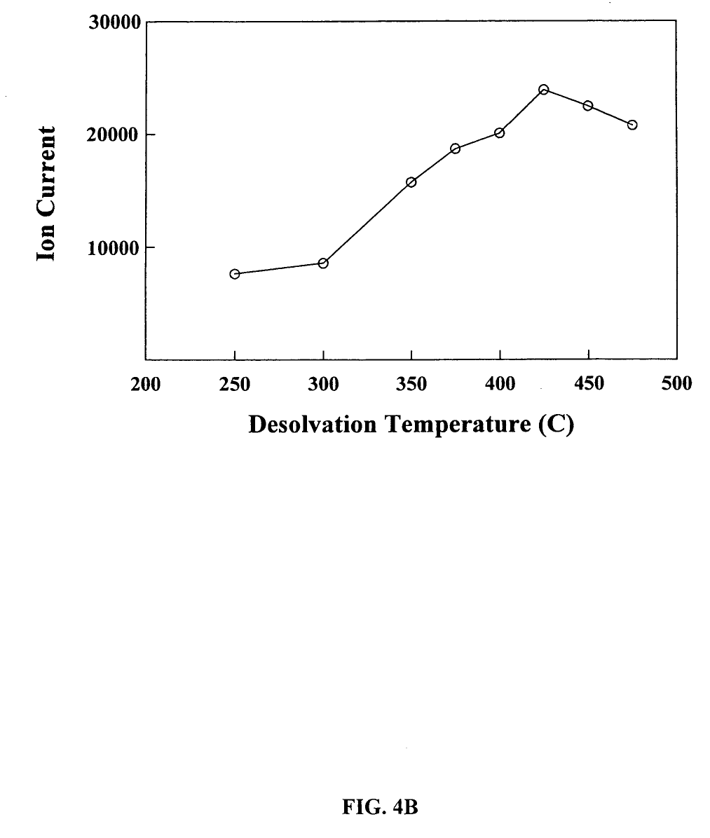

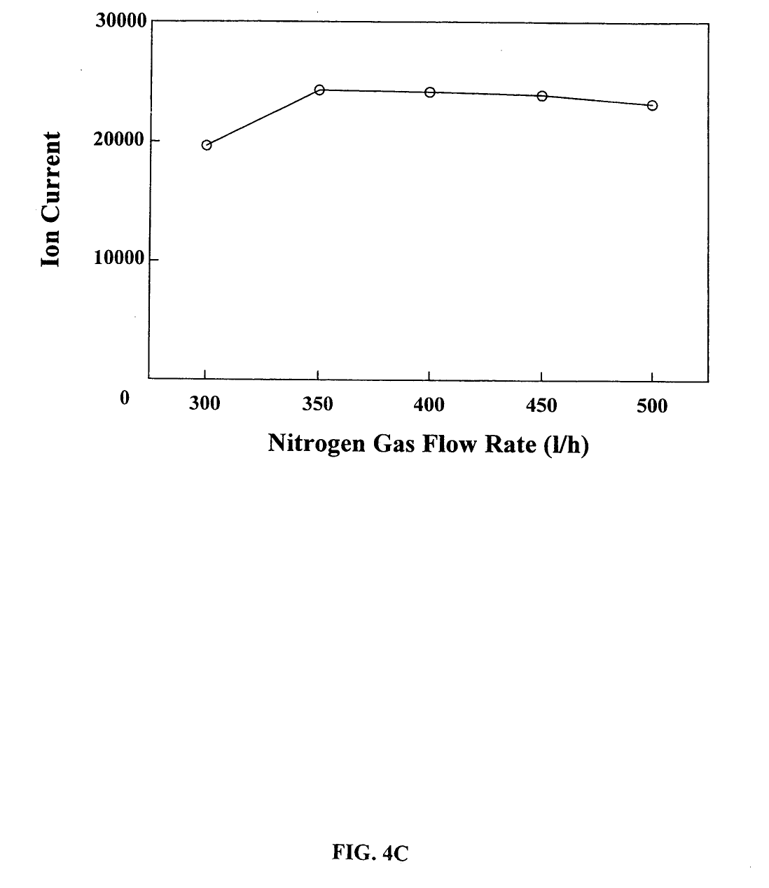

In order to facilitate the measurement of therapeutic agents such as voriconazole concentration in aqueous humor with high sensitivity, the ESI interface parameters were optimized. Summarized in FIG. 4A, FIG. 4B, and FIG. 4C are the effects of cone voltage, desolvation temperature, and nebulizer nitrogen gas flow rate on the intensity of the protonated voriconazole molecular ion (m/z=350). An aliquot of 0.3 ng voriconazole was injected for each test, and the ion intensity was measured in the SIR mode. The highest ion intensity was achieved when the cone voltage was set to 10 volts (FIG. 4A). When the cone voltage was increased higher than 15 volts, the ion current signal decreased significantly. The ion current intensity (FIG. 4B) was enhanced with increasing desolvation temperature up to 425° C. Further increases in desolvation temperature (from 425 to 475° C.), however, caused the depression of the ion current signal. The maximum ion intensity was achieved at 350 l/h for nebulizer nitrogen gas flow, and was not increased at a higher N₂ flow rate (FIG. 4C).

Under optimized mass detector conditions, linearity was assessed over the range from 0.04 ng to 10 ng of injected voriconazole. A calibration curve was constructed with data obtained from injection of six different amounts of voriconazole over this range. A linear function was determined, with the following equation:

Peak Area=63994×(ng voriconazole injected)+6478.4 (1)

The coefficient of regression of this equation was 0.9989. Detailed accuracy data obtained by analysis of a set of voriconazole standard samples spiked in blank aqueous humor are listed in Table I. The coefficients of variance (CV) were less than 3% over the whole analytical range, and the deviations were less than 5%. Table II illustrates the efficiency of voriconazole recovery (mean plus standard deviation (SD)) at concentrations of 75, 300, 750 μg/l. The recovery of voriconazole from aqueous humor averaged 95% (Table II). The intra-day and inter-day imprecision of voriconazole determination for samples of 60, 200 and 800 μg/l was less than 3% (Table III).

TABLE I
ACCURACY OF THE LC-ESI-MS METHOD

| Theoretical | Measured concentration | | | |
|---|---|---|---|---|
| concentration (μg/l) | (Mean ± SD, μg/l) | CV (%) | Accuracy (%) | Deviation (%) |
| 60 | 58.59 ± 1.38 | 2.36 | 97.65 | −2.35 |
| 200 | 208.67 ± 3.09 | 1.48 | 104.34 | 4.33 |
| 500 | 519.55 ± 12.48 | 2.40 | 103.91 | 3.91 |
| 800 | 790.60 ± 9.67 | 1.22 | 98.83 | −1.18 |
| 1000 | 962.36 ± 6.09 | 0.63 | 96.24 | −3.76 |

n = 4;
SD, standard deviation;
CV, coefficient of variance.
Accuracy was expressed as a percentage of the mean measured concentration over the theoretical concentration.

TABLE II
RECOVERY (%) OF VORICONAZOLE (N = 4)
FROM AQUEOUS HUMOR

| Concentration (μg/l) | Recovery (% ± SD) |
|---|---|
| 75 | 94.07 ± 2.63 |
| 300 | 95.89 ± 3.51 |
| 750 | 94.92 ± 3.12 |

TABLE III
IMPRECISION OF THE LC-ESI-MS METHOD

| | |
|---|---|
| Theoretical | Imprecision (%) |

| concentration (μg/l) | Intra-day | Inter-day |
|---|---|---|
| 60 | 1.44 | 2.88 |
| 200 | 0.48 | 1.76 |
| 800 | 0.16 | 1.45 |

Imprecision was expressed in terms of coefficient of variation (n=4).

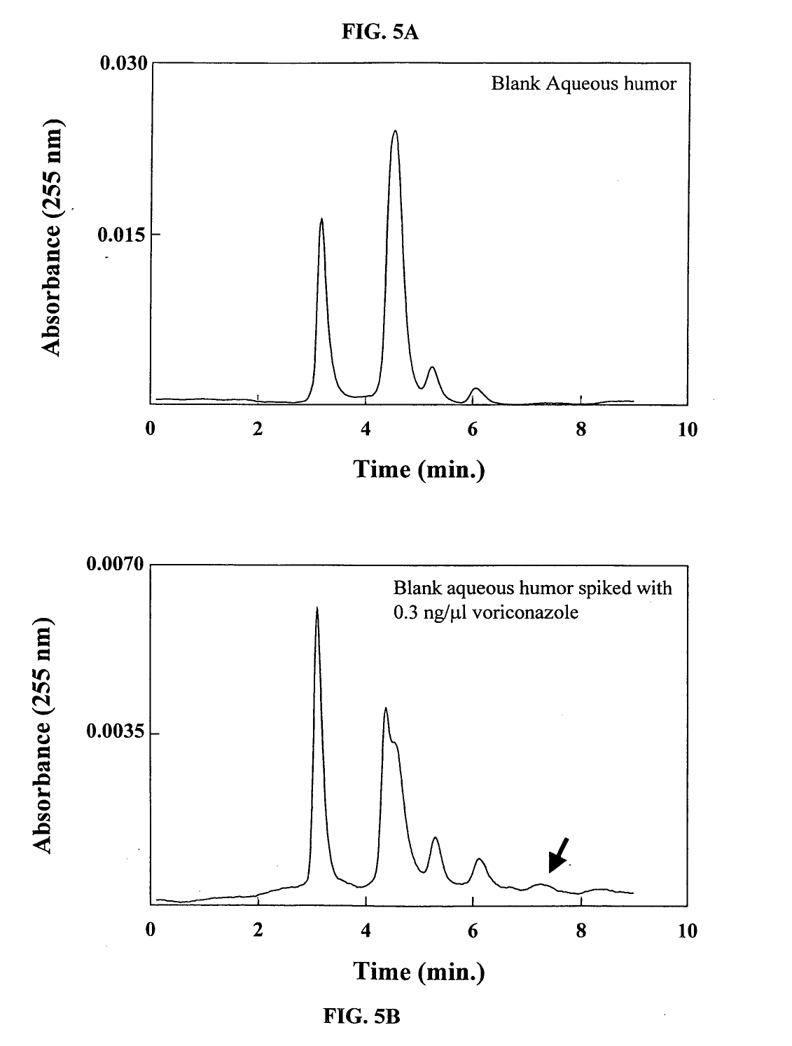

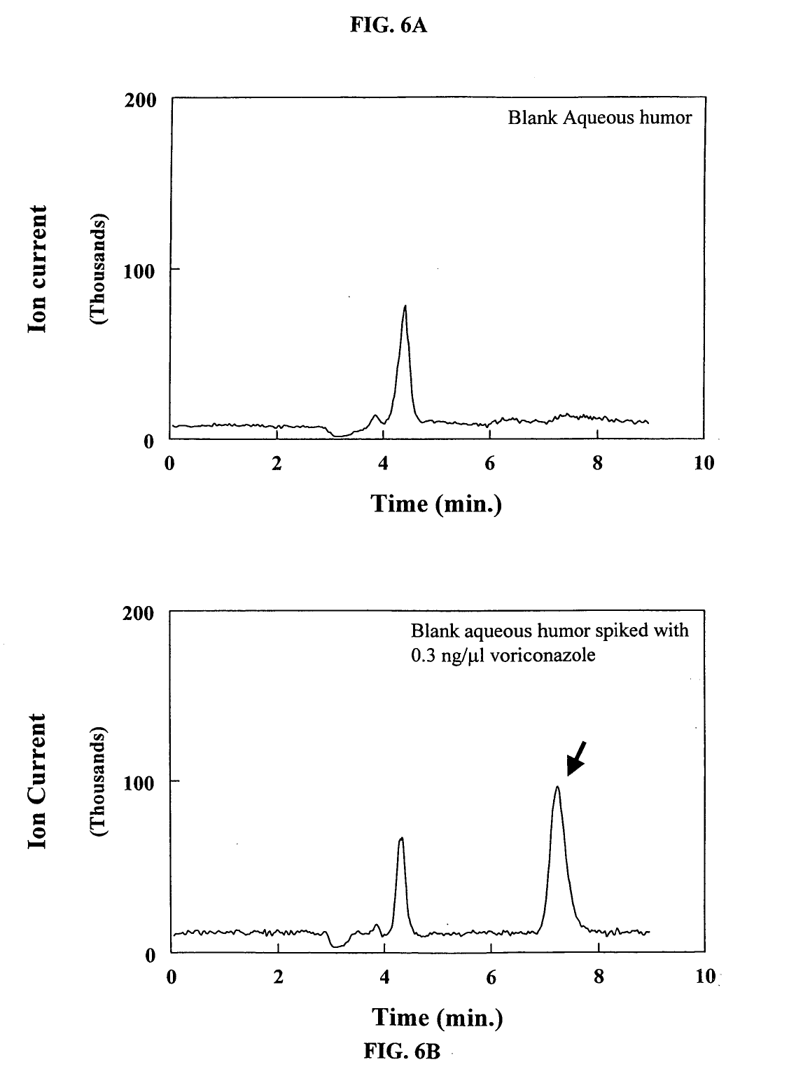

Typical UV (255 nm) chromatograms obtained for blank aqueous humor and blank aqueous humor spiked with 0.3 ng/μl voriconazole are shown in FIG. 5A (blank) and FIG. 5B (spiked sample). There is no detectable voriconazole peak in FIG. 5B (the amount injected into the column was 0.6 ng). The sensitivity of HPLC with UV detection (255 μm) is not high enough to detect the voriconazole in this case. The limit of detection of voriconazole using HPLC with UV detection (255 nm) is about 5.0 ng/assay (0.05 ng/μl, injected in a volume of 100-μl) (Gage and Stopher, 1998). The corresponding SIR chromatograms (m/z=350) for blank aqueous humor (FIG. 6A) and blank aqueous humor spiked with 0.3 ng/μl voriconazole (FIG. 6B) clearly demonstrate a voriconazole peak (RT=7.2 min, indicated by an arrow). Another peak, seen in both FIG. 6A and FIG. 6B with RT of 4.1 min, appeared in both blank aqueous humor and aqueous humor spiked with voriconazole; therefore, it is not a voriconazole peak, but apparently is a component in aqueous humor with the same ion size of m/z=350. As it separates well from the voriconazole peak (RT=7.2), it did not interfere with the analysis.

The limit of detection using the LC-MS method is estimated to be 10 pg, at a signal-to-noise ratio of three. Hence, it can be used to determine the penetration of voriconazole into aqueous humor at a concentration as low as 0.1 ng/ml, which is a detection limit more than 500 times as sensitive as that of HPLC with UV detection, reported as 0.05 μg/ml by Gage and Stopher (1998). The practical threshold for routine HPLC-UV measurement of voriconazole, however, may be considerably higher, starting at 0.2 μg/ml (Perea et al., 2000).

When voriconazole analyses by LC-ESI-MS and by HPLC-UV were compared, the two methods correlated very well. A regression analysis of LC-ESI-MS against HPLC-UV found the following relation:

[ng Voriconazole]_{LC-ESI-MS}=0.9717×[ng Voriconazole]_{HPLC-UV}-0.0793 (2)

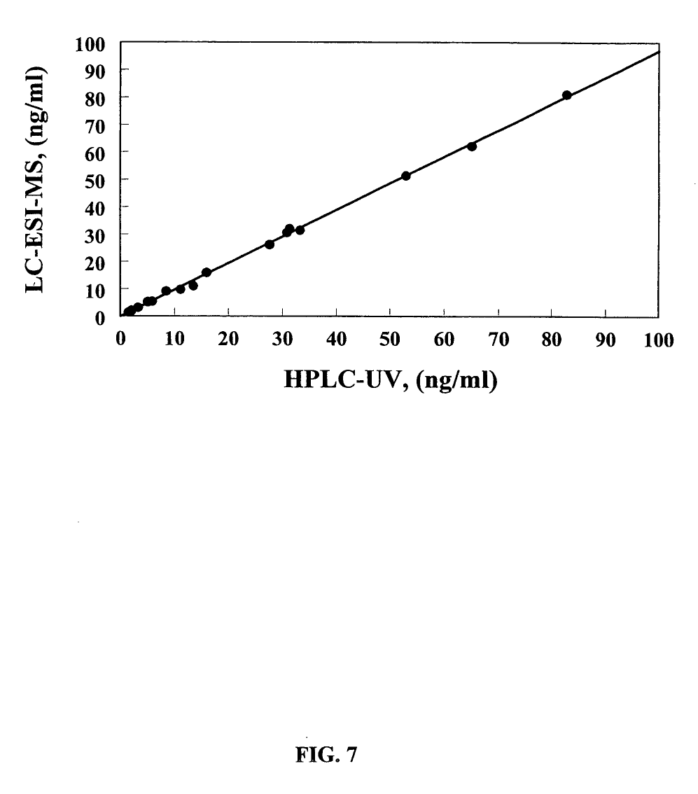

The regression coefficient was 0.9985. This correlation is shown graphically in FIG. 7.

As a practical validation, LC-ESI-MS was used to determine the aqueous humor concentration of voriconazole in the rabbit eye after topical application twice daily of 5 μg or 10 μg of voriconazole for eleven days. The voriconazole concentration in aqueous humor from animals receiving the lower dose was 7.34±5.88 ng/ml (n=4), whereas in the animals receiving the higher dose, the concentration was 14.7±12.99 ng/ml (n=7). The UV and SIR chromatograms of the aqueous humor samples in treated eyes were very similar to those shown in FIG. 5B and FIG. 6B.

The utility of the LC-ESI-MS method for pharmacokinetic studies of therapeutic agents such as voriconazole in animal or human investigations may be appreciated by comparing the sensitivity of this method to the reported minimum inhibitory concentration (MIC) of therapeutic agents such as voriconazole against various fungal and related organisms. The Aspergillus family is considered to be especially suitable for treatment with voriconazole. For A. fumigatus, the MIC is approximately 0.2 μg/ml, and for other Aspergillus species the MIC ranges from 80 ng/ml to 0.8 μg/ml (Murphy et al., 1997; Wildfeuer et al., 1998). The MIC for Candida species has been reported to range from 1 ng/ml to 0.4 μg/ml (Kappe, 1999; Marco et al., 1998). In general, the MIC for yeasts and molds is on the order of 0.5-0.6 μg/ml (Murphy et al., 1997; Marco et al., 1998).

Based on the foregoing in vitro observations, a clinically useful blood plasma concentration of 1.2 to 4.7 μg/ml has been recommended (Perea et al., 2000). The inventors contemplate that the effective doses of this and other agents for treatment of ocular infections may be lower than the recommended plasma levels. While the clinically relevant plasma concentration of therapeutic agents such as voriconazole can be monitored by HPLC-UV, ocular treatment levels may be below the practical sensitivity of UV detection, but are well within the working range of LC-ESI-MS. Therefore, LC-ESI-MS, because of its greater sensitivity, is better suited to measuring tissue penetration of such drugs during pharmacokinetic investigations.

The methodological aspects of the present invention therefore provide techniques based on LC-ESI-MS to determine the concentration of therapeutic agents such as voriconazole in aqueous humor. The sensitivity, selectivity, and the rapidity exceeded those of HPLC-UV methods. The analytical methods developed in the invention are thus well suited to the study of the pharmacokinetics of therapeutic agents such as voriconazole in animals and humans.

### EXAMPLE 2

### Topical Treatment for Fungal Keratitis

The present example establishes that the topical administration of antimicrobials is potentially of use in the treatment of infections, such as fungal keratitis. The example uses an accepted animal model in rabbits, and shows that topical voriconazole is potentially of use in the treatment of P. lilacinus keratitis in rabbit eyes.

Paecilomyces lilacinus is a common soil-dwelling mould, which occasionally has been associated with human disease. Although it is an uncommon cause of corneal infection, it can produce a devastating keratitis leading to endophthalmitis and loss of the eye. There have been over a dozen cases reported of infection (e.g., endophthalmitis, orbital granuloma, sinusitis, cutaneous mycosis) caused by P. lilacinus in the literature. Most cases of P. lilacinus in the literature have been associated with surgical procedures or use of nonsterile solutions. Treating infections caused by this organism is very challenging given its inherent resistance to traditional antifungal agents including amphotericin B and natamycin. Prior to the present invention, no standard antifungal regimen had been demonstrated to provide an effective cure for this type of infection.

The inventors were referred a patient with a central corneal ulcer caused by P. lilacinus. The patient was initially treated by other practitioners, using a combination of amphotericin B and steroids. Cultures of the ulcer grew P. lilacinus sensitive only to ketoconazole. The inventors commenced treatment with topical ketoconazole and systemic terbinafine, which prevented further circumferential expansion of the lesion. Despite this, the cornea ultimately perforated, necessitating urgent penetrating keratoplasty. Cultures of the cornea and the aqueous of this patient were positive for P. lilacinus, and isolates from these were used for the studies in the present example.

Voriconazole is a broad-spectrum antifungal agent used extensively in the systemic treatment of Aspergillus and Candida species (Barry et al., 1996; Denning et al., 1996; 1997; Marco et al., 1998; Ghannoum et al., 1999; Kirkpatrick et al., 2000; Lozano-Chiu et al., 2000). The efficacy of topical voriconazole in the treatment of P. lilacinus keratitis was studied in rabbit eyes. The penetration of topically applied voriconazole into the cornea, conjunctiva, aqueous, iris, chorioretina and vitreous was also evaluated.

A. Materials and Methods

1. Pathogen

Paecilomyces lilacinus # 00-39 is a clinical isolate from an infected cornea obtained from the Fungus Testing Laboratory at the University of Texas Health Science Center in San Antonio, Tex. The isolate was maintained on Sabouraud's agar. Prior to inducement of corneal infection, the moulds were cultured on sporulation agar to induce formation of conidia. The infected plates were overlaid with sterile water, and conidia were separated from mycelia using a spinning magnetic bar. Conidia were further separated from mycelial fragments by filtration through nylon wool, and then counted in a hemacytometer.

2. In vitro Susceptibility

In vitro susceptibility was determined for voriconazole using the National Committee for Clinical Laboratory Standards method modified for mycelial pathogens. The minimum inhibitory concentration of voriconazole for isolate P. lilacinus #00-39 was 0.25 μg/ml 24 hours after inoculation, and 0.5 μg/ml 48 hours after inoculation.

3. Animals

New Zealand white rabbits, average weight 2-3 Kg, were maintained at the University of Texas Health Science Center Medical School Animal Unit. Animals were maintained in compliance with the Association for Research in Vision and Ophthalmology Resolution on the Use of Animals in Research.

4. Drug

Voriconazole (UK-109496) powder with 99.9% purity was obtained directly from Pfizer Central Research. It was suspended in 0.3% Noble Agar to a concentration of 5 μg/ml.

5. Infection

Rabbits were anesthetized and inoculated with conidia of P. lilacinus. Corneas were infected under an operating microscope. A 1.5 mm triangular lamellar midstromal dissection into the right central cornea was performed using the tip an Alcon 3.0 mm keratome. The internal apex of the wound was further extended intrastromally using a 27-gauge cannula on a tuberculin syringe, which dissected a further 4 mm narrow intrastromal pocket. Then 0.02 ml (3.2×10⁷) of the conidial suspension was injected under the flap and into the pocket via the 27-gauge cannula.

6. Monitoring Growth and Inclusion Criteria

A pilot group of 5 rabbits was infected and not treated, to assess the natural growth pattern of the corneal ulcers in an untreated but infected eye when the contralateral eye received the drug. This model would thus provide a basis for isolating the antifungal efficacy of topically applied drug, independent of any vascular delivery of voriconazole which might arise as a consequence of systemic crossover from the fellow eye. The resulting lesions were observed twice daily, at 10 am and 4 pm. Within 3 days, all 5 untreated corneal lesions had attained a diameter of 2 mm in either the vertical or horizontal axis.

Treatment with voriconazole in subsequent studies was therefore initiated when either the vertical or horizontal diameter of the P lilacinus lesion had reached 2 mm. Narcotic analgesics were administered subcutaneously to control pain and minimize discomfort to the animals throughout all studies.

7. Treatment

Topical voriconazole suspended in Noble Agar was given twice a day to one eye of each animal. The animals were divided into 3 groups, as follows:

Pilot group: As stated above, 5 rabbits were infected in the right eye only. No voriconazole was administered to the infected right eye. The non-infected left eye received two separate 5 μg doses of topically applied voriconazole daily, one at 10 am and the other at 4 pm.

5 Treatment Group: Five rabbits were infected intracorneally with Paecilomyces lilacinus in the right eye only. The eyes were observed twice daily until a lesion of 2 mm diameter, either vertically or horizontally, was present in the infected eye. The infected right eye was thereafter treated topically with 5 μg voriconazole in 0.1 ml noble agar twice daily, at 10 am and 4 pm. The non-infected left eye received a single drop of 0.1 ml of the sterile noble agar vehicle as a control at each treatment session.

10 μg Treatment Group: 5 rabbits were infected intracorneally with Paecilomyces lilacinus in the right eye only, and were treated with 10 μg topical voriconazole to that eye. Voriconazole was applied twice daily, at approximately 10 am and 4 pm. The left eye was administered 0.1 ml of noble agar as a control at each session.

Pilot group rabbits were observed for a total of 3½ days, by which time all had lesions attaining a diameter of ≧2 mm in either the horizontal or vertical dimension. The two treatment groups (5 μg and 10%1 g) were observed for 11 days in total. Treatment of the infected eye with voriconazole was initiated when corneal ulcer attained a diameter of at least 2 mm in either the horizontal or vertical dimension, typically between day 2 and day 4. Measurement of the corneal infiltrate was made using a ruler and a 4× magnifier.

8. Dissection

All animals were euthanized using intravenous Sodium Pentobarbital 100-150 mg/kg. Both eyes were dissected with initial removal of a 1×1 cm of conjunctiva from the lower nasal corner of the eye. Aqueous was retrieved using a 30-gauge needle on a tuberculin syringe. The cornea, iris, vitreous and chorioretina were then placed into different tubes and immediately stored in ice. The samples of conjunctiva, cornea, iris, and retina were then weighed on a Mettler electronic microbalance.

9. Extraction

In the past, a multidimensional high-performance liquid chromatography was developed for the analysis of voriconazole in human plasma. Base on this idea, a novel method using liquid chromatography-electrospray ionization mass spectrometry (LC-ESI-MS) has been developed for analysis of voriconazole in aqueous humor in conjunction with this study (Example 1). The separation was achieved on a reversed-phase C18 column eluted with 70% acetonitrile and 0.01% trifluoroacetic acid against 30% water with 0.01% trifluoroacetic acid. The correlation between the concentration of voriconazole to peak area was linear (R²=0.998) between 0.04 to 10 ng. The coefficient of variance was less than 3%. The limit of determination was estimated to be 0.1 ng/ml voriconazole in aqueous humor. This technique is 500 times more sensitive than the conventional HPLC-UV detection method. The intra-day and inter-day imprecision were both less than 3% over the whole analytical range

10. Microbiology

Half of the dissected tissues (i.e. cornea, conjunctiva, iris) and all of aqueous humor of the randomized animals were sent for microbiology. The samples were homogenized and serial dilution counts made from <5 colony forming units (CFU) per specimen. These were plated out and counted to evaluate for growth of the fungus.

B. Results and Discussion

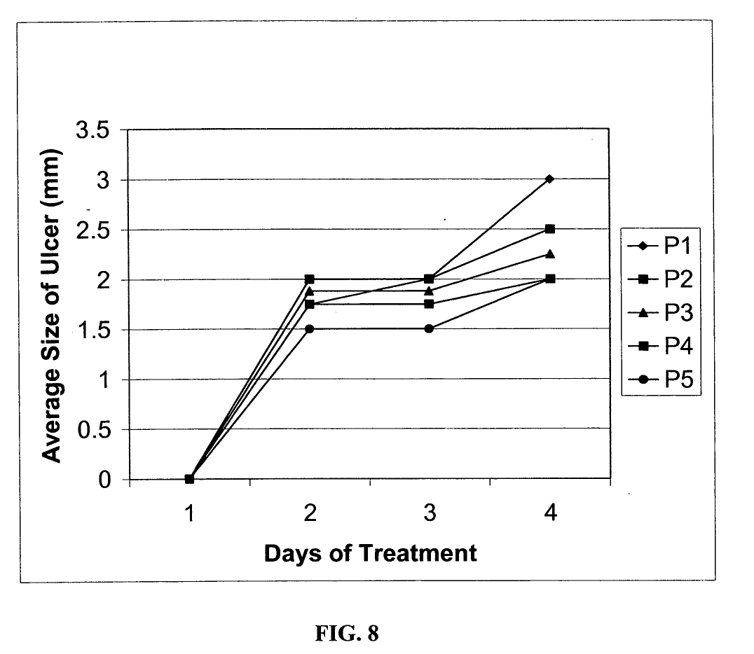

In the Pilot Group (FIG. 8), there was a latency of about one day after inoculation. Ulceration was negligible on day 1 in the infected eye, but marked ulceration was evident by day 2.5. There was progressive increase in the size of the ulcer in the 5 infected eyes, all receiving no direct treatment. Additionally, there was marked conjunctival inflammation and hypopyon noted in all five infected eyes. No apparent inhibition of growth was conferred systemically by the twice-daily 5 μg topical doses of voriconazole to the non-infected contralateral eye. This group of rabbits was euthanized on day 3.5 to determine the amount of voriconazole in ocular tissues and fluids of the treated, uninfected eyes. The application of medication to the control eyes produced no observable changes in the appearance of their conjunctiva or cornea.

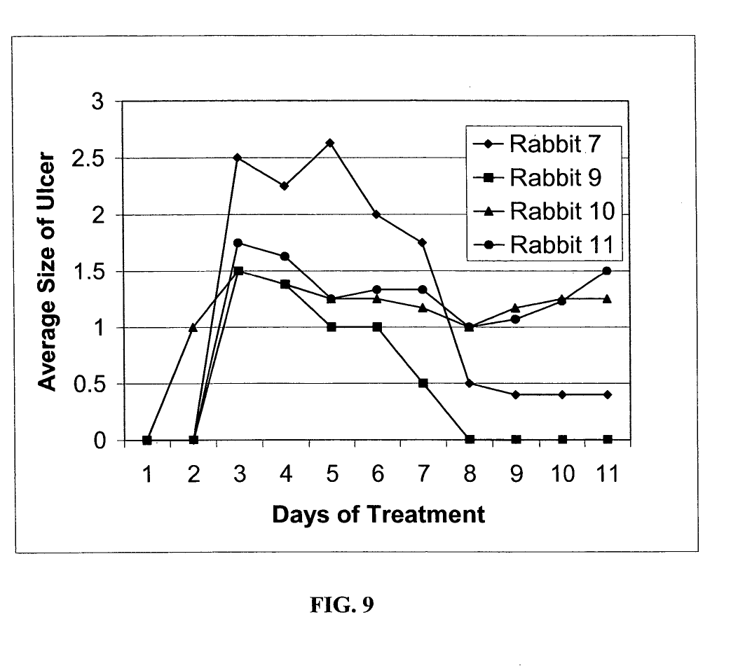

In the 5 μg Treatment Group (FIG. 9), there was an initial increase in maximal diameter (vertical or horizontal) of the ulcers, after inoculation and prior to treatment with voriconazole, to 1.5-2.5 mm by day 3. Four rabbits commenced therapy on the morning of day 3. After starting twice-daily topical treatment, the ulcer size in the cornea of two rabbits (#10 and 11) decreased from a mean (horizontal+vertical diameter/2) of 1.5 mm down to 1 mm between days 3 and 5. Their ulcers remained about the same size through day 11.5, until sacrifice. One animal (#9) achieved a total cure of the ulcer (i.e. no visible infiltrate in the cornea) after 5.5 days (i.e. by day 8.5). The ulcer of another animal (#7) was reduced to below 0.5 mm by day 7. Rabbit #8 failed to produce a corneal lesion of ≧2 mm in either dimension, and was therefore excluded from further analysis.

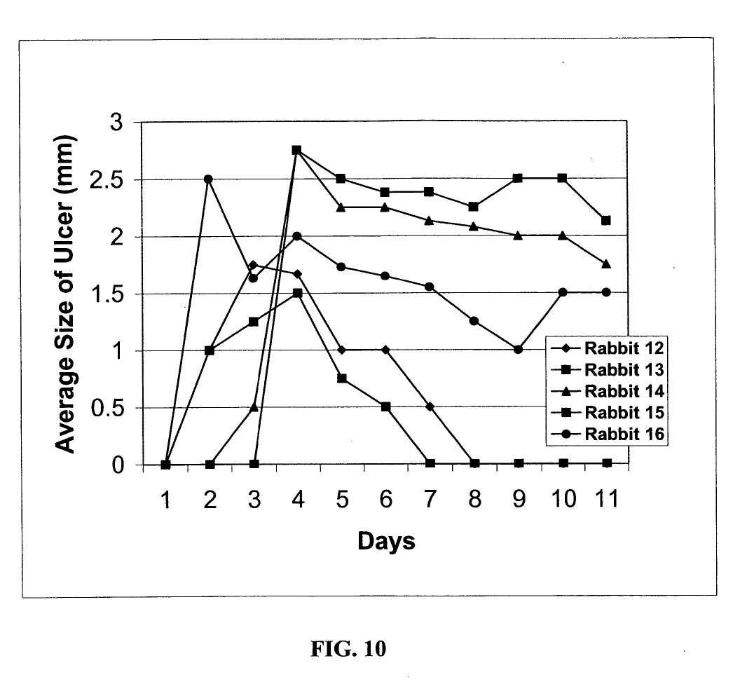

The 10 μg Treatment Group (FIG. 10) also demonstrated a significant initial increase in ulcer size prior to voriconazole therapy. A steady decrease in the size of the ulcer in all 5 animals occurred after initiation of treatment on day 3. Two animals (#12,13) achieved total cure after 7.5 days. The ulcer size of the other 3 eyes (#14,15,16) remained about the same up to 11.5 days, at completion of the study.

The distribution of voriconazole in the cornea, conjunctiva, iris, retina, aqueous humor, and vitreous was determined in the 3 study groups. Voriconazole concentration was highest in the conjunctiva in each group. The standard deviation and the standard error of the mean for each group were determined. There were substantial variations of drug concentration in the cornea and conjunctiva in all groups, with correspondingly high standard deviation values for these superficial ocular tissues.

In assessing the clinical course, FIG. 8 shows the natural growth rate of intracorneal lesions for the Paecilomyces among the pilot group, demonstrating progression of fungal growth to a mean lesion diameter of ≧2 mm in all five infected corneas, in the absence of treatment, by day 3. Examples of retardation and regression of corneal lesions are evident when voriconazole treatment was applied in both the 5 μg (FIG. 2) and 10 μg (FIG. 3) treatment groups.

Microbiology counts verified that the fungal infection was present in the inoculated corneas of all but one animal (#7), which did, however, demonstrate fungal growth in the conjunctiva and iris.

The histopathology of random samples of the infected corneal tissue in the Pilot group, 5 μg treatment group and 10 μg treatment group was assessed. There was evidence of the isolated fungal infection seen within the corneal stroma of the Pilot group. In the two treatment groups (5 μg and 10 μg), there were only inflammatory cells, few fungal elements, and corneal scarring identified.

The data appear to demonstrate voriconazole's capacity to inhibit progressive corneal ulceration by P. lilacinus. Ulceration was arrested at or below 2 mm size, or reversed, in all treated eyes, within a short treatment period.

Voriconazole concentration was highest in the conjunctiva. Voriconazole is almost insoluble in water. On immediate inspection after topical application, the voriconazole suspension was not evenly distributed throughout the cornea and conjunctiva. This irregular surface distribution of voriconazole on the conjunctiva appears to account for variations in the concentration of voriconazole obtained among the corneal and conjunctival specimens. In contradistinction, despite extremely high focal concentrations in the conjunctiva and cornea of some eyes, the variations of voriconazole concentration in the retina, iris, aqueous humor and vitreous were relatively small.

Clearly, the cornea and conjunctiva harbor a significant proportion of the voriconazole applied, but drug penetration into the eye also occurs. Statistically significant higher concentrations of voriconazole were observed in the conjunctiva than in all other tissues among the pilot group (all values P<0.05). Mean corneal concentrations were also significantly higher than in the retina (P=0.03) and vitreous (P=0.04) in this group. Furthermore, among infected eyes treated for 10 days, twice daily, with 5 μg of voriconazole, mean conjunctival concentrations significantly exceeded those in all other intraocular compartments (all values P<0.001). Similar trends were evident in both eyes of each treatment group, with corneal and conjunctival concentrations tending to exceed intraocular concentrations by at least one order of magnitude. There were no significant differences in voriconazole concentration between the intraocular tissues within any group or between treatment groups, with mean concentrations ranging between 1.7 and 37.3 μg/ml. Despite the visible superficial loculation and variations in conjunctival concentration, topical voriconazole still gains access deep into the eye.

In summary, voriconazole, an unmodified powder suspension, can penetrate into and throughout intraocular tissues and demonstrates effectiveness in treating corneal keratitis caused by Paecilomyces lilacinus. Its penetration and fairly uniform intraocular distribution indicate that this drug will be of value in the treatment of P. lilacinus and other related fungal endophthalmitides, particularly if increasing concentrations can be achieved in the eye. As voriconazole has a broad antifungal spectrum, and is effective against treating ocular pathogens such as Candida, Aspergillus, and Fusarium, this medication has many potential applications in topical treatment regimens.

### EXAMPLE 3

### Clinical Treatment With Topical Anti-Fungal

The present inventors have treated resistant human fusarium endophthalmitis by topically applying a triazole suspension approved for oral use. The patient was a 42 year old woman who previously had 20/20 uncorrected vision but contracted a fungal keratitis from a cosmetic contact lens. Her infection had progressed rapidly from a central corneal lesion to endophthalmitis, throughout an intensive two-month course of natamycin, amphotericin B, and imidazole treatment by the referring team of ophthalmologists.

Under an investigative new drug FDA-approved compassionate use protocol, the inventors administered a combination of topical and systemic posiconazole (SCH56592; Schering Health Care, Sussex UK). Despite eventual surgical biopsy revealing evidence of fungal mycelia in the cornea, iris and anterior capsule, stabilization and recovery of vision were accompanied by positive aqueous triazole levels (plasma 1.6 μg/ml; aqueous 0.9 μg/ml; using Bodet's modification of the yeast nitrogen base agar bioassay). The relatively high aqueous levels suggest penetration of the topically administered posiconazole, and its likely role in her recovery from an infection which seemed likely to result in enucleation of the eye on standard therapy.

### EXAMPLE 4

### Prostanoids Potentiate Ocular Drug Penetration

The present example is a first pilot study demonstrating that prostanoids, such as prostaglandins, potentiate the effects of ocular therapeutic agents by improving their penetration. This is particularly exemplified by the use of the PGF2α analogue, latanoprost, to enhance ocular permeability to the antimicrobial drug voriconazole, administered subconjunctivally. This study therefore extends the data of Example 2 and Example 3 by providing surprisingly effective vehicles for the improved delivery of antimicrobials and other drugs into the eye.

Rabbits are selected in these studies as they are particularly valuable animal models because of the similarity of the structures of rabbit eyes to human eyes. The chosen model allows evaluation of the natural progression of disease and its response to the new anti-fungal medication in vivo. Density of infection, fungal growth and amount of drug penetration into the intraocular tissues can be analyzed, whereas in vitro susceptibility studies have not been indicative of in vivo efficacy and do not account for tissue distribution of the drug, or required dosing regimens for effective and long term treatment of fungal infections in a biological system. These requirements are met by the present model.

Two New Zealand rabbits were given one drop of latanoprost (Xalatan™; Pharmacia & Upjohn) OD daily for five days. The control left eye was given one drop of saline daily for five days. On day 6, the rabbits were sedated and a single 0.03 ml dose of 5 mg/ml methylcellulose suspension (150 μg) of voriconazole (Pfizer) was injected via 30 gauge needle into the subconjunctiva of both eyes. The animals were sacrificed after 70 minutes. The four eyes were dissected and the aqueous analyzed by liquid chromatography-electronspray ionization mass spectrometry (LC-ESI-MS), using the method of Example 1, to determine the concentration of voriconazole.

These studies showed the concentrations of voriconazole in the aqueous humour of the latanoprost-treated eyes of the two rabbits to be 19.5 ng/ml and 25.5 ng/ml, respectively. No trace of the medication could be detected in either of the contralateral eyes, which had received identical concomitant subconjunctival doses of voriconazole.

Comparative studies showed that the concentrations of voriconazole in the aqueous humor of the latanoprost-pretreated eyes of these acutely-treated rabbits (after 70 minutes) were comparable to those obtained after greater than 8 days of twice-daily topical administration of 10 μg of voriconazole among 5 other New Zealand rabbits.

Using latanoprost as a prostaglandin and voriconazole as an ocular therapeutic agent, the present example therefore shows that prostanoids augment ocular drug penetration. Clearly, the penetration of voriconazole was enhanced in the eyes receiving topical pre-administration of latanoprost. Although not being bound by any mechanistic theories, the present inventors believe that latanoprost could function by increasing the permeability of the ciliary muscle and the blood-aqueous barrier to allow the observed improved and expedited penetration of pharmacological agents into the aqueous and vitreous humors. The collagenase-induction effects of latanoprost might also contribute to the resultant enhanced ocular permeability.

### EXAMPLE 5

### Subconjunctival Coadministration of Prostanoids Potentiates Ocular Drug Penetration

This example extends the first pilot study of Example 4, providing additional data on the use of subconjunctival administration of prostanoids, such as prostaglandins, to potentiate the penetration of ocular therapeutic agents, as exemplified by the anti-fungal agent voriconazole.

In light of the above pilot study, the present example was conducted to show that effective tissue concentrations of voriconazole could be attained more rapidly, with a single dose of the antifungal, by pretreating eyes with a prostanoid.

One drop of latanoprost 0.0005% was instilled into the right eyes of four New Zealand rabbits for 5 days, and one drop of 0.9% saline administered into the left eyes for the same time period. The animals were sedated on day 6, and 0.03 ml (150 μg) of voriconazole (Pfizer) in a 5 mg/ml methylcellulose solution was subconjunctivally injected into both eyes. After 70 minutes, the aqueous humour was removed from both eyes via paracentesis. Residual proteins were removed from the samples by solid phase extraction, eluted with methanol, and evaporated to dryness. They were reconstituted with a mobile phase of 70% acetonitrile, 1% acetic acid to protonate the voriconazole, and 30% water. The aqueous samples were then analyzed by liquid chromatography-electrospray ionization mass spectrometry (LC-ESI-MS) to determine the amount of voriconazole. Voriconazole has a M.W. of 349 and the protonated form has a M.W. of 350.

In 3 of the 4 eyes pre-treated with latanoprost, LC-MS analysis of the aqueous sample gave a mass spectrum at the retention time, 7.2 minutes, which showed a quantifiable peak at 350. This corresponded to the molecular ion of a voriconazole standard (M.W. 349, M+1=350). No mass peak at 350.1 was detected at the appropriate retention time in 3 of the 4 left eyes that received an equal dose of voriconazole without latanoprost pretreatment. One of the left eyes not pretreated with latanoprost had a small peak corresponding to a detectable but nonquantifiable level (<0.5 ng/ml) of voriconazole. In the four latanoprost pre-treated eyes, the concentrations of voriconazole were calculated to be 0, 1.5, 33 and 75 ng/ml, based on comparisons to a standard voriconazole calibration curve.

Although the number of subjects is small, the paired eye results from this study show a pronounced effect of latanoprost pre-treatment on the ocular penetration of voriconazole. This study therefore validates the pilot study and supports the use of topical prostaglandins and analogues to enhance penetration of another applied drug. Both concurrent application and pre-treatment with prostanoids are contemplated for use in increasing the amount of antimicrobials and other drugs penetrating the ocular tissue.

### EXAMPLE 6

### Topical Coadministration of Prostanoids Potentiates Ocular Drug Penetration

The present example further develops the studies of Example 4 and Example 5, this time providing data on the topical administration of prostanoids, such as prostaglandins, to enhance the penetration of ocular therapeutic agents, as exemplified by the anti-fungal agent voriconazole.

In addition to further validating the present invention, these data include some particularly important features. Notably, that the drug levels are even higher with the present topical application than with the subconjunctival bolus of methylcellulose/drug combination of the previous examples. In addition, voriconazole shows excellent penetration into the vitreous with topically co-applied latanoprost. This is important as drug penetration into the posterior chamber poses the most significant clinical problem, which can now be effectively overcome, as shown by the increased drug penetration into the vitreous.

A. Materials and Methods

1. Pharmacological Agents

Voriconazole was obtained from Pfizer (New Haven, Conn.) as a dry powder. It was formulated as an ophthalmic topical agent by suspending the powder in noble agar at 5 μg/ml. Latanoprost, 50 μg/ml, was obtained as Xalatan™ ophthalmic solution from Pharmacia & Upjohn (Kalamazoo, Mich.).

2. Administration of Drugs

The studies were carried out in New Zealand White rabbits. All animal procedures were conducted in accordance with the ARVO Statement for the Use of Animals in Ophthalmic and Vision Research. The rabbits were divided into two groups. In the control group, two animals were anesthetized by subcutaneous injection of 1-2 ml/kg of “rabbit cocktail” (a mixture of 100 mg/ml ketamine, 100 mg/ml xylazine, and 25 mg/ml acepromazine), and a single drop of voriconazole (VCZ) was instilled topically into both eyes of two animals. At 3.5 hr after VCZ administration, samples were obtained from the eyes with 1-ml syringes fitted with 25-ga needles for the aqueous samples, and 21-ga needles for the vitreous samples. Sample volumes averaged 125±28.9 μl for aqueous humor, and 387.5±184.3 μl for vitreous humor.

In the experimental group, 5 rabbits were pretreated with one drop of latanoprost (LT) to the right eye, and one drop of saline to the left eye, once per day for six days. On day six, the rabbits were anesthetized by injection of 1-2 ml/kg of rabbit cocktail, and a single drop of VCZ suspension was placed in the right eye only. Aqueous and vitreous samples were taken as in the control group except that samples were obtained at 2 hr in two rabbits, and 3.5 hr in three rabbits. Average aqueous and vitreous sample volumes in the experimental group were 179±48.7 μl and 396±154.6 μl, respectively. Following the collection of the samples, the rabbits were euthanized by i.v. injection of 100-150 mg/kg sodium pentobarbital.

3. Sample Preparation

Proteins and other large molecules were removed from the samples by solid phase extraction (SPE) through Oasis HLB SPE cartridges (Waters, Milford, Mass.). Prior to introduction of the sample, the cartridges were conditioned by passage of 1 ml methanol and 1 ml water, in that order. The volume of the sample was recorded, and then the sample was loaded into the SPE cartridge. The cartridge was washed with 1 ml of water, and then eluted with 1 ml of methanol. The eluate was evaporated to dryness, and the residuum was stored at −70° C. until analysis.

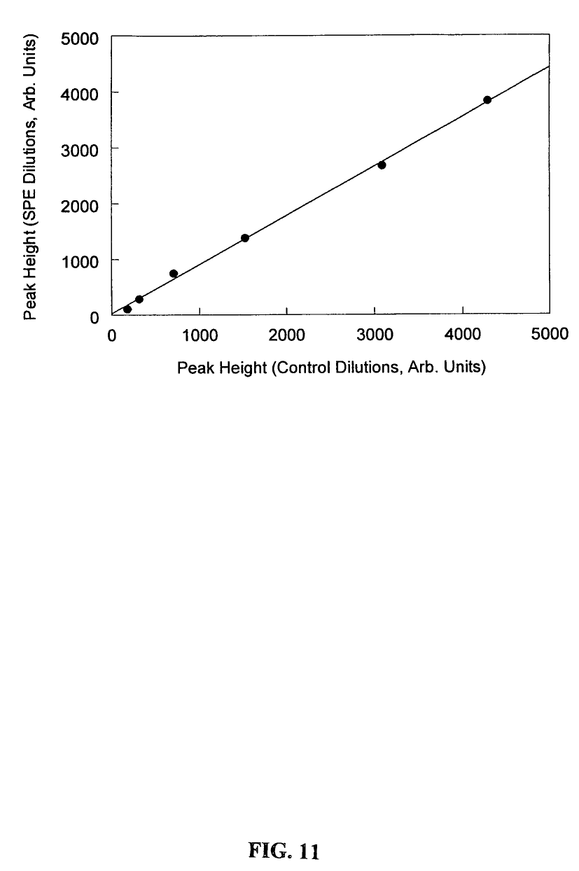

The recovery efficiency of the extraction step was determined by extracting six dilutions of authentic VCZ through SPE cartridges, and then measuring the amount of VCZ present by HPLC analysis. In order that the sample matrix of these standard dilutions resembled that of the physiological samples, the standards were prepared by making aqueous dilutions of a 1 mg/ml stock solution of VCZ dissolved in methanol. Six dilutions in water over the range of 50 ng/ml to 1,500 ng/ml were used. A similar dilution series, not subjected to SPE, was also measured with HPLC, and the results compared (FIG. 11). The correlation (slope) of the extracted to unextracted standards was 0.88 (r²=0.9989), indicating that the mean recovery efficiency of VCZ with SPE was 88%.

4. Analysis of Voriconazole by Liquid Chromatography-Mass Spectrometry

The amount of VCZ contained in the aqueous and vitreous samples was measured using liquid chromatography-mass spectrometry (LC-MS), on a Finnegan MAT ThermaQuest™ LCQ electrospray ionization mass spectrometer with an ion trap detector. The chromatographic separation was performed with a Waters Spherisorb™ 4.6×150 mm analytical column. The mobile phase was 70% acetonitrile-1% acetic acid in water, and the flow rate was 0.5 ml/min. Desiccated samples were reconstituted in 200 μl of mobile phase for injection into the chromatographic column. The relative concentration or dilution of the sample (the ratio of the original sample volume before SPE to the final, 200 μl sample volume) was noted and the measurements made with the LC-MS were adjusted accordingly.

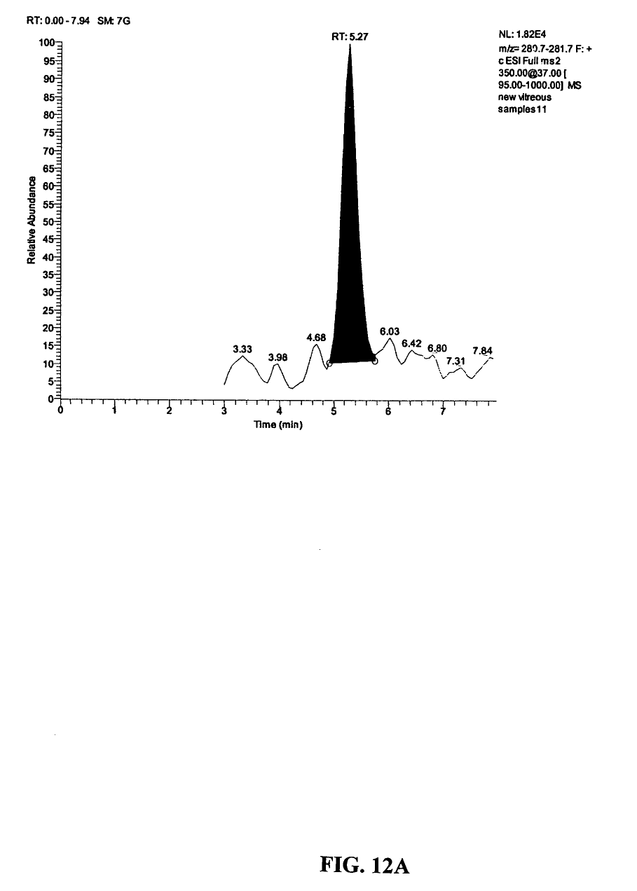

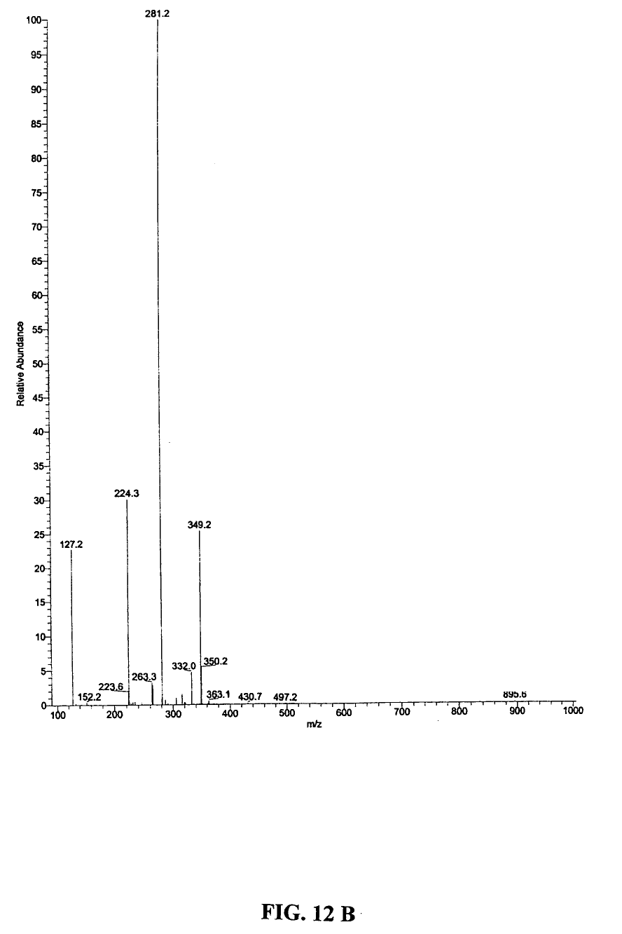

All sample injections were made by an autoinjector at a consistent volume of 10-μl. Under these conditions, the retention time of VCZ was approximately 5.3 min (FIG. 12A). The protonated parent molecule ([M+]) of VCZ has a mass to charge ratio (m/z) of 350.2. For analysis, however, the VCZ molecule was fragmented by increasing the cone voltage of the electrospray ionization unit until two consistent daughter fragments were found at m/z=281.2 and 224.3 (FIG. 12B). Due to the presence of a confounding peak at 349.2, and to increase the specificity of the LC-MS assay, the quantitation of VCZ in the samples was based on the magnitude of the 281.2 daughter fragment instead of the parent molecule.

5. Calibration

Calibration of the instrument was done using a dilution series of authentic VCZ standards. In order to compensate for recovery efficiency of the SPE sample preparation step, the VCZ standards had been previously processed by SPE and resuspended in mobile phase prior to injection into the LC-MS. Calibration was achieved over the range of 100 pg to 10 μg of injected VCZ, corresponding to 10-μl injections of the standard dilutions over the concentration range of 10 ng/ml to 1000 ng/ml). Over this range, the response of the mass spectrometer for the 281.2 m/z daughter fragment was linear with an r-value of 0.992. Injections of less than 100 pg VCZ produced a response that deviated excessively from the linear calibration line, and were classified as below the level of quantitation. The limit of detection was estimated to be about 10 μg, which corresponded to about 1 ng/ml with the present preparative and analytical methods.

B. Results and Discussion

Samples obtained from eyes of two groups of animals were analyzed. The experimental group contained five animals that received LT pretreatment for a week in one eye followed by a single dose of VCZ, and a week of saline treatment in the fellow eye. The control group contained two animals that received VCZ only (no pretreatment) in both eyes. In the experimental group, the data from one of the rabbits was excluded because of cross-contamination of the samples during the collection procedure. This reduced the number of eyes in each arm of the experimental group to four. The calculated VCZ concentrations shown below have been adjusted from the actual LC-MS measurements on the basis of the relative concentration or dilution of the samples by the SPE/reconstitution process.

The effect of sampling time on VCZ penetration was shown to be insignificant, by the following reasoning. In the two experimental groups, i.e., the LT+VCZ-treated eyes and the saline-treated eyes, aqueous and vitreous samples were collected variously at 2 hrs and 3.5 hrs post treatment. This time difference, however, was found to be insignificant in terms of VCZ penetration (p>0.05, two-tailed Mann-Whitney test). Therefore, in the subsequent statistical analyses of the results, the sample time was ignored, and the data from the eyes sampled at the two times were pooled.

The aqueous penetration of voriconazole was first measured. The measurements of the individual samples are shown in Table IV.

TABLE IV
PENETRATION OF VCZ INTO THE AQUEOUS COMPARTMENT

| | | |
|---|---|---|
| Sample ID | Sample Time (hrs) | Tissue Conc. (ng/ml) |

Control Group: Single topical dose of VCZ only, both eyes

| | | |
|---|---|---|
| L21A | 3.5 | 128.4 |
| L22A | 3.5 | 192.1 |
| R21A | 3.5 | 124.0 |
| R22A | 3.5 | 930.2 |

Exp. Group¹: (O.D.) LT-pretreatment (6 days) followed by single
topical dose of VCZ

| | | |
|---|---|---|
| R1A | 2.0 | 479.1 |
| R2A | 2.0 | 246.2 |
| R4A | 3.5 | 155.6 |
| R5A | 3.5 | 398.6 |

Exp. Group¹: (O.S.) Saline treatment only

| | | |
|---|---|---|
| L1A | 2.0 | 17.1 |
| L2A | 2.0 | ²N.Q. |
| L4A | 3.5 | 45.1 |
| L5A | 3.5 | ²N.Q. |

¹Samples from one rabbit (R3, not shown) were excluded because of cross-contamination during handling.
²N.Q. = not quantifiable, i.e., VCZ was detected, but at amounts below the limit of quantitation (<10 ng/ml)

Accounting for all the values in Table IV, in eyes that received a single dose of VCZ, but no LT pretreatment, the drug concentration in the aqueous was 343.7±392.3 ng/ml. The average aqueous VCZ concentration in eyes that received LT pretreatment plus VCZ was 319.8±146.2 ng/ml. In eyes that received only saline, the VCZ concentration was found to be 16.1±20.8 ng/ml. Both VCZ-treated eyes had amounts of drug that differed significantly from the saline treated eyes (p<0.05, Bonferroni multiple comparison test), but not from each other. These data in total therefore appear to show that VCZ penetrates the aqueous compartment quite readily, and that LT pretreatment does not appear to enhance the penetration of the drug into the aqueous.

However, if the 930.2 value in the control (single dose of VCZ, but no LT pretreatment) is not counted, then the mean of three values in the control group without pretreatment is 148.2. The mean of the four values in the pretreatment group, 319.8, is thus higher than the re-calculated control. By discounting the highest value in each of the control (930.2) and pretreatment groups (479.1), the mean of three values in the pretreatment group is 266.8, which is higher than the corresponding 148.2 of the control, although the remaining number of variates (3) in the group make it difficult to assess the statistical significance of the result in this meta-analysis. The vitreous data, as discussed below, are clear cut and require no meta-analysis.

The vitreous penetration of voriconazole was next measured, and the individual sample measurements are shown in Table V.

TABLE V
PENETRATION OF VCZ INTO THE VITREOUS COMPARTMENT

| | | |
|---|---|---|
| Sample ID | Sample Time (hrs) | ²Tissue Conc. (ng/ml) |

Control Group: Single topical dose of VCZ only, both eyes

| | | |
|---|---|---|
| L21 V | 3.5 | 20.3 |
| L22 V | 3.5 | N.Q. |
| R21 V | 3.5 | N.Q. |
| R22 V | 3.5 | N.Q. |

Exp. Group¹: (O.D.) LT-pretreatment (6 days) followed by single
topical dose of VCZ

| | | |
|---|---|---|
| R1 V | 2.0 | 24.8 |
| R2 V | 2.0 | 115.0 |
| R4 V | 3.5 | 169.2 |
| R5 V | 3.5 | 35.7 |

Exp. Group¹: (O.S.) Saline treatment only

| | | |
|---|---|---|
| L1 V | 2.0 | N.Q. |
| L2 V | 2.0 | N.Q. |
| L4 V | 3.5 | N.Q. |
| L5 V | 3.5 | N.Q. |

¹Samples from one rabbit (R3, not shown) were excluded because of cross-contamination during handling.
²N.Q. = not quantifiable, i.e., VCZ was detected, but at amounts below the limit of quantitation (<10 ng/ml)

As shown in Table V, the VCZ concentration in the vitreous of eyes that received a single dose of the drug without LT pretreatment could be quantified in only one eye, which had a concentration of 20.3 ng/ml VCZ. The other three eyes had detectable but not quantifiable amounts of VCZ, which indicated that the effective concentration of VCZ in those eyes was less than 10 ng/ml. The average VCZ concentration in the vitreous of eyes that had received a week's pretreatment of LT was 86.2±59.2 ng/ml., and the amount of VCZ in every eye of this group was well above the limit of quantitation. In the eyes that received only saline, none of the four eyes had an amount of VCZ in the vitreous that exceeded the limit of quantitation. In order to perform a statistical analysis on these results, eyes that had detectable but not quantifiable amounts of VCZ were arbitrarily assigned a concentration of drug corresponding to the limit of detection, i.e., 1 ng/ml. With these assignments, the LT-pretreated group had a significantly greater amount of VCZ in the vitreous compared to either the VCZ-only group (p˜0.014, Bonferroni) or saline-only group (p˜0.019, Bonferroni).

Over time, PGF2α can cause structural or metabolic changes demonstrated by the eventual loss of extracellular material between the bundles of ciliary muscle. This will lead to widening of the spaces between the bundles. Relaxation of the ciliary muscle is one of the consequences of PGF2α administration. It is possible that either of these actions contribute to the increased penetration of the antimicrobial. A direct action on the sclera is also apparent.

Irrespective of the mechanism of action, the present invention therefore shows that the application of prostaglandins, such as latanoprost, enhances the penetration of other applied drugs, as exemplified by the antimicrobial voriconazole used in the present study.

### EXAMPLE 7

### Topical Coadministration of Prostanoids Potentiates Ocular Drug Penetration

The present inventors have shown the efficacy and penetration of topical voriconazole in the treatment of fungal keratitis caused by Paecilomyces lilacinus, and that latanoprost administration with topical voriconazole significantly increases the penetration of the antifungal drug into the intraocular tissues. Although the previous examples adequately demonstrate the invention, additional studies using topical voriconazole and latanoprost to treat Aspergillus endophthalmitis further exemplify the important applications of the present invention. Studies that compare the efficacy of the combination medication versus the standard drug of choice, Amphotericin B, in treating Aspergillus are particularly useful. High-performance Liquid Chromatography-Mass spectrometry assays are used to determine the penetration of both drugs in the intraocular tissues and histopathology is used to evaluate the final pathological outcome.

Aspergillus is a common fungal pathogen that can causes a devastating endophthalmitis. Despite the standard of treatment at present with intravitreal Amphotericin B injection, it is still very difficult to treat. Moreover, Amphotericin B brings about many toxicities to the intraocular tissues. Voriconazole, in vitro, has demonstrated a higher potency than amphotericin B against Aspergillus and less toxicity. Moreover, the previous examples show that topical voriconazole demonstrates very good intraocular penetration when coadministered with subconjunctival injection of latanoprost.

Pars plana intravitreal injections of the fungus into the right eye of each rabbit are performed and observed for growth. The animals are properly sedated and anesthetized with intramuscular ketamine HCL (40 mg/Kg) and topical proparacaine as needed before inoculation. All animals receive antifungal medication in at least one eye once adequate endophthalmitis is confirmed. The natural process of endophthalmitis and its resolution after treatment is monitored by indirect ophthalmoscopy and ERG monitoring.

The use of one pilot group and two treatment groups (voriconazole treatment group and amphotericin treatment group) is advantageous. The pilot group contains 5 rabbits. These rabbits receive an intravitreal injection of Aspergillus into the right eye. The left eye serves as a control. Endophthalmitis is confirmed based on indirect ophthalmoscopy on the infected eye, grading vitreous haze on a standard scale of 1-4. Serial ERG is performed on both eyes every 6-12 hrs after inoculation.

The Voriconazole treatment group contains 15 rabbits. The right eye of each rabbit receives an intravitreal injection of Aspergillus. ERG is performed on both eyes of the sedated rabbits prior to inoculation as a baseline. Treatment with either topical voriconazole and topical latanoprost, intravenous voriconazole, or intravitreal voriconazole is randomly given to each rabbit approximately 1-2 days after inoculation when active endophthalmitis have been confirmed. The rabbits are sacrifice at the end of the study (approximately 8-10 days after treatment has started) and samples of their intraocular contents from both eyes are randomly sent for histopathology, microbiology, and HPLC-analysis

The Amphotericin B treatment group also contains 15 rabbits. The right eye of each rabbit receives an intravitreal injection of Aspergillus. ERG is performed on both eyes prior to inoculation. Treatment with either topical amphotericin B and topical latanoprost, intravenous amphotericin B, or intravitreal amphotericin B is given to the infected eye of each rabbit approximately 1-2 days after inoculation when active endophthalmitis have been confirmed. At the end of the study, approximately 8-10 days after treatment has started, the rabbits are sacrificed and their intraocular contents of both eyes sent for histopathology, microbiology, and HPLC-analysis.

Pain control is given throughout. Indirect ophthalmoscopy and ERG is performed under sedation. The animals are treated according to the standards of the Association for Research in Vision and Ophthalmology resolution on the use of animals in research. Resolution or improvement of the eye infection occurs in all treated eyes.

For statistical analyses, as in any other study within the range of the embodiments of the present invention, nonparametric methods (Mann Whitney test) are useful for comparisons of tissue counts, as there are occasional outlying values. Similar tests are used for measuring the size of the corneal lesions followed on a daily basis, and for comparison of tissue concentrations. P<0.05 determines significance.

Efficacious voriconazole treatment for Aspergillus is a better choice than current therapy, as it causes less toxicity to the intraocular tissues. Moreover, the topical usage of the present invention will be more preferred, being as efficacious as other route of administering medication, yet providing a safer and easier way of treating fungal endophthalmitis.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods, and in the steps or in the sequence of steps of the methods described herein, without departing from the concept, spirit and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.

AREDS Report No. 8, Arch Ophthalmol., 119:1417-1436, 2001.

Barry A L, Brown S D. Antimicrobial Agents and Chemotherapy, 40:1948-1949, 1996.

Brem, Angiogenesis, 2: 9-20, 1998.

Bu, Khan, Poglod, Micetich, J. Chromatogr. B Biomed. Sci. Appl., 724(2):311-317, 1999.

Denning D, Del Favero A, Gluckman E, Norfolk D, Ruhnke M, Yonren S, Troke P, Sarantis S, Thirty Fifth Interscience Conference on Antimicrobial Agents and Chemotherapy, F80: 126 (Abstract), 1996.

Denning D, Del Favero A, Gluckman E, Norfolk D, Ruhnke M, Yonren S, Troke P, Sarantis S, 13^{th} Congress of the International Society for Human and Animal Mycology, Abstract P552:217 (Abstract), 1997.

Dickison, Bell, Hitchcock, Narayanaswami, Ray, Richardson, Troke, Bioorg. Med Chem. Lett., 6(16):2031-2036, 1996.

Gage and Stopher, J. Pharm. Biomed. Anal.,17:1449-1453, 1998.

Ghannoum, Okogbule-Wonodi, Bhat, Sanati, J. Chemother., 11(1):34-39, 1999.

Hegener, Troke, Fatkenheuer, Diehl, Ruhnke, AIDS, 12(16):2227-2228, 1998.

Kappe, Mycoses, 42(Suppl. 2):83-86, 1999.

Kirchner, Vidal, Winkler, Mueller, Jacobsen, Franzke, Sewing, Ther. Drug Monit., 21(1):116-122, 1999.

Kirkpatrick W R, Mcatee R K, Fothergill A W, Rinaldi M G, Patterson T F, Antimicrobial Agents and Chemotherapy, 44: 2865-2868, 2000.

Lozano-Chiu M, Arikan S, Paetznick V L, Anaissie E J, Rex J H, J. Clin. Microbiol., 37: 2755-2759, 1999.

Marco, Pfaller, Messer, Jones, Antimicrob. Agents Chemother., 42(1):161-163, 1998.

Martin, Yates, Hitchcock, Antimicrob. Agents Chemother., 41(1):13-16, 1997.

Matsuura, Kuwano, Takashina, J. Chromatogr., 621:173-180, 1993.

Muller, et al., Structure, 6(9):1153-67, 1998.

Murphy, Bernard, Ishimaru, Armstrong, Antimicrob. Agents Chemother., 41(3):696-698, 1997.

Osborne et al., Exp. Eye Res., 69(3):331-342, 1999.

Perea, Pennick, Modak, Fothergill, Sutton, Sheehan, Rinaldi, Antimicrob. Agents Chemother., 44(5):1209-1213, 2000.

Presta, Chen, O'Connor, Chisholm, Meng, Krummen, Winkler, Ferrara, Cancer Res., 57:4593-4599, 1997.

Seddon, Ajani, Sperduto, Hiller, Blair, Burton, Farber, Gragoudas, Haller, Miller, et al., JAMA, 272(18):1413-20, 1994.

Siriwardena, Khaw, King, Donaldson, Overton, Migdal, Cordeiro, Ophthalmology, 109(3):427-31, 2002.

Stopher and Gage, J. Chromatogr. B Biomed Sci. Appl., 691(2):441-448, 1997.

van't Hek, Verweij, Weemaes, van Dalen, Yntema, Meis, Am. J Resp. Crit. Care Med, 157(5 Pt 1):1694-1696, 1998.

Wildfeuer, Seidl, Paule, Haberreiter, Mycoses, 41(7-8):309-319, 1998.

1. A method of increasing the intraocular amount of a biological agent, comprising providing to an eye of an animal at least a first biological agent and an amount of at least a first prostanoid effective to increase the intraocular amount of said at least a first biological agent.

2-105. (canceled)

106. The method of claim 1, wherein at least one of said at least a first biological agent and said at least a first prostanoid are provided to said eye by topical administration.

107. The method of claim 1, wherein at least one of said at least a first biological agent and said at least a first prostanoid are provided to said eye by subconjunctival injection.

108. The method of claim 1, wherein said at least a first biological agent and said at least a first prostanoid are comprised within a single composition.

109. The method of claim 1, wherein said at least a first biological agent and said at least a first prostanoid are comprised within distinct first and second compositions.

110. The method of claim 109, wherein at least a first composition comprising said at least a first prostanoid is provided to said eye at a biologically effective time prior to at least a second composition comprising said at least a first biological agent.

111. The method of claim 109, wherein the composition comprising said at least a first prostanoid is provided to said eye by topical administration and wherein the composition comprising said at least a first biological agent is subsequently provided to said eye by subconjunctival injection.

112. The method of claim 1, wherein the intraocular amount of said at least a first biological agent is increased in the aqueous of said eye.

113. The method of claim 1, wherein the intraocular amount of said at least a first biological agent is increased in the vitreous of said eye.

114. The method of claim 1, wherein at least a second biological agent is further provided to said eye.

115. The method of claim 1, wherein at least a second prostanoid is further provided to said eye.

116. The method of claim 1, wherein said at least a first prostanoid is a prostaglandin.

117. The method of claim 116, wherein said at least a first prostanoid is a PGD or PGF prostaglandin or derivative thereof.

118. The method of claim 117, wherein said at least a first prostanoid is a phenyl-substituted, 3-oxa or 3-carba analog of a PGD or PGF prostaglandin.

119. The method of claim 117, wherein said at least a first prostanoid is a PGD₂ or PGF2α analogue.

120. The method of claim 119, wherein said at least a first prostanoid is Latanoprost™.

121. The method of claim 1, wherein said at least a first biological agent is a detectable or diagnostic agent.

122. The method of claim 1, wherein said at least a first biological agent is a therapeutic agent.

123. The method of claim 122, wherein said at least a first biological agent is dilating agent, a mydriatic, a cycloplegic, a miotic or a cholinesterase inhibitor.

124. The method of claim 122, wherein said at least a first biological agent is an agent selected from the group consisting of phenylephrine, tropicamide, cyclopentolate, homatropine, scopolamine and atropine.

125. The method of claim 122, wherein said at least a first biological agent is a steroid, a non-steroidal anti-inflammatory agent, anti-histamine, anti-glaucoma agent, topical carbonic anhydrase inhibitor, cholinesterase inhibitor, topical beta adrenergic blocking agent, topical alpha adrenergic agonist, reducing agent, anti-oxidant, vitamin, mineral, growth factor, immune modulator, cytokine, hormone, antibody, anesthetic, anti-neoplastic agent.

126. The method of claim 122, wherein said at least a first biological agent is an anti-microbial, anti-bacterial, anti-viral, anti-retroviral, anti-parasitic or an anti-fungal agent.

127. The method of claim 126, wherein said at least a first biological agent is an anti-microbial agent selected from the group consisting of neomycin, polymyxin B, erythromycin, trimethoprim, sulfacetamide sodium, tetracycline, oxytetracycline, norfloxacin, ciloxan, ciprofloxacin, levafloxacin, ofloxacin, gentamycin, tobramycin, vancomycin, bacitracin, cephazolin, amikacin, ketoconazole, trifluridine, caspofungin, amphotericin B and natamycin.

128. The method of claim 126, wherein said at least a first biological agent is a glycopolypeptide, a macrolide, a beta lactam, an aminoglycoside or a quinolone anti-bacterial agent.

129. The method of claim 126, wherein said at least a first biological agent is the anti-viral agent ganciclovir, acyclovir, famciclovir, foscarnet or cidofovir.

130. The method of claim 126, wherein said at least a first biological agent is the anti-fungal agent voriconazole.

131. The method of claim 122, wherein said at least a first biological agent is a protein, polypeptide or peptide.

132. The method of claim 122, wherein said at least a first biological agent is DNA, RNA, an antisense construct, a ribozyme, or a plasmid or viral expression vector or recombinant virus containing such a virus.

133. The method of claim 1, wherein said at least a first prostanoid is Latanoprost™ and wherein said at least a first biological agent is voriconazole.

134. The method of claim 1, wherein said animal has or is suspected of having an allergy or allergies affecting the eye, diabetes, glaucoma, ocular neovascular disease, retinal degeneration, macular degeneration or a vitamin deficiency that affects the eye.

135. The method of claim 1, wherein said animal has or is suspected of having an optic neuropathy, a blunt or penetrating ocular injury or an orbital or intraocular tumor.

136. The method of claim 1, wherein said animal has or is suspected of having a microbial, bacterial, viral, retroviral, parasitic, fungal or amoebal infection.

137. The method of claim 136, wherein said animal has or is suspected of having an acute infection, chronic infection, preseptal cellulitis, orbital cellulitis or periorbital cellulitis.

138. The method of claim 136, wherein said animal has or is suspected of having an HIV-, CMV- or HSV-associated retinal disorder, a gram positive bacterial infection, a gram negative bacterial infection, candidiasis, aspergillosis, bacterial or fungal keratitis or endophthalmitis, uveitis, conjunctivitis or an intraocular or periocular inflammation.

139. The method of claim 1, wherein said animal has or is preparing to undergo eye surgery.

140. The method of claim 139, wherein said animal is preparing to undergo eye surgery and wherein said at least a first biological agent is an anesthetic.

141. The method of claim 139, wherein said animal has been subjected to eye surgery and wherein said at least a first biological agent is an anti-microbial, anti-bacterial, anti-viral, anti-retroviral, anti-parasitic or an anti-fungal agent.

142. The method of claim 1, wherein said animal is a human subject.

143. A method of increasing the amount of voriconazole in the intraocular space of the eye, comprising contacting an eye of an animal with a combined effective amount of voriconazole and Latanoprost™; wherein said Latanoprost™ increases the amount of voriconazole in the intraocular space of said eye in comparison to the amount of voriconazole in the intraocular space of said eye in the absence of said Latanoprost™.

144. An ophthalmically acceptable formulation comprising an ocular-transport effective amount of at least a first prostanoid and a therapeutically effective amount of at least a first ophthalmically active biological agent.

145. A kit comprising, in at least a first suitable container, a therapeutically effective combination of at least a first prostanoid and at least a first ophthalmically active biological agent.
